(19) United States
(12) Patent Application Publication
(10) Pub. No.: US 2009/0053295 A1
(43) Pub. Date: Feb. 26, 2009

(54) REGULATABLE FUSION PROMOTERS

(76) Inventor: Charles Stout, Loma Linda, CA (US)

Correspondence Address: FISH & RICHARDSON PC, P.O. BOX 1022, MINNEAPOLIS, MN, 55440-1022, US

(21) Appl. No.: 12/088,921

(22) PCT Filed: Sep. 29, 2006
(86) PCT No.: PCT/US06/38154

§ 371 Date: Oct. 9, 2008

Related U.S. Application Data
(60) Provisional application No. 60/722,568, filed on Oct. 1, 2005.

(51) Int. Cl. A61K 9/127; C12N 15/11; C12N 15/00; C12N 5/08; C12N 5/00; C12N 5/04; C12Q 1/68; A61P 43/00; A61K 35/76; C12N 1/19; A01K 67/027; A01H 5/00; A61K 31/7088

(52) U.S. Cl. 424/450; 536/24.1; 435/320.1; 435/366; 435/348; 435/419; 435/254.11; 800/13; 800/298; 514/44; 435/349; 435/350; 435/351; 424/93.2; 435/375; 435/6

(57) ABSTRACT

Fusion promoters are described that combine a RNA polymerase III basal promoter and regulatory elements from RNA polymerase II regulatory regions, and which provide specific regulation of expression from the promoter. Such fusion promoters are useful, for example, for expressing RNAi agents in vivo.

### RELATED APPLICATIONS

This application claims the benefit of an earlier-filed provisional application, US. Ser. No. 60/722,568, filed Oct. 1, 2005, the content of which is hereby incorporated by reference in its entirety.

### FIELD OF THE INVENTION

The present invention relates to RNA polymerase promoters for targeted and/or regulated transcription of coding sequences, and in particular for expressing RNA sequences for RNA interference (RNAi), micro RNA (miRNA), aptamers, short interfering RNA (siRNA), and/or short hairpin RNA (shRNA).

### BACKGROUND OF THE INVENTION

The following discussion is provided solely to assist the understanding of the reader, and does not constitute an admission that any of the information discussed or references cited constitute prior art to the present invention.

Short RNA duplexes of approximately 18 to 30 base pairs have been shown to initiate several types of sequence-specific regulation of gene expression. In one type of regulation, i.e. RNA interference (RNAi), these short RNA duplexes cause sequence-selective degradation of mRNA in a wide range of eukaryotic cells, including mammalian cells. In one embodiment of RNAi, small interfering RNAs (siRNAs) are about 21 nucleotides (nt) long and paired such that they have a 19 base pair stem and 2-nt 3′-overhanging ends that, when introduced to eukaryotic cells, cause sequence-selective degradation of targeted mRNA and gene suppression (Caplen, et al. (2001) Proc Natl Acad Sci USA, 98, 9742-9747; Elbashir, et al. (2001) Nature, 411, 494-498). In another embodiment of RNAi, in vivo transcription of DNA constructs delivered into eukaryotic cells is utilized to introduce: 1) long dsRNAs which are enzymatically processed resulting in short dsRNAs, 2) small hairpin RNAs (shRNAs) or, 3) separate short complementary strands that can hybridize in vivo to form siRNA. The short dsRNA duplexes delivered by any of the mentioned methods trigger degradation of target RNAs mediated by incorporation of one of the strands in a RNA-induced silencing complex (RISC). It has been observed that double-stranded RNA longer than 30 base pairs can activate the interferon response causing nonspecific tranlational arrest and apoptosis.

Another type of regulation of gene expression by short RNA duplexes involves a class of genes that encode short dsRNA hairpin loops of about 24 to 30 basepairs in length that are processed to about 21 to 23 nt small RNAs. These short RNA duplexes, termed micro RNAs (miRNAs), function in the same pathway as siRNAs by associating with Argonaute proteins that are required for guiding target mRNA recognition. mRNAs cleave complementary target mRNAs in plants but appear to repress mRNA translation rather than mRNA cleavage in animals.

Another category of functional short RNAs, termed aptamers or intramers, are RNAs that are 23 to 400 nucleotides in length that display high affinity and selectivity towards a diverse array of targets, including both proteins and small molecules. Binding of aptamers to the target protein or molecule can block or otherwise modulate molecular function. Riboswitches are natural RNA aptamers involved in genetic regulation.

siRNA, shRNA, RNAi, RNA aptamers, e.g., riboswitches, and miRNA (termed short RNAs throughout this document) can be introduced into cells via classic gene transfer methods such as liposome-mediated transfection, electroporation, calcium shock, hydrodynamic shock or microinjection which requires chemical or enzymatic synthesis of siRNAs prior its application. They can also be generated intracellularly by transcription from plasmid DNA, integrated transgene loci, or retroviral, lentiviral or adenoviral constructs. Intracellular transcription of small RNA molecules is possible by cloning the siRNA templates into RNA polymerase III (pol III) transcription units, which normally encode the small nuclear RNA U6 or the human Rnase P RNA H1.

Typically, shRNAs are synthesized from vectors (e.g., plasmids or viral vectors). Generally, such synthesis is driven by type III RNA polymerase (Pol III) promoters. Pol III promoters are generally ubiquitous. A commonly used Pol III promoter is the U6 promoter, a strong constitutive promoter. In general, Pol III produces small, non-coding transcripts such as U6 small nuclear RNA (snRNA), which are not capped at the 5′ and not polyadenylated at the 3′ end. Pol III promoter elements include a distal sequence element (DSE), proximal sequence element (PSE), and TATA box, located 5′ to the initiation site. Additionally, transcription driven by Pol III promoters initiates at defined nucleotides, terminates when the transcription encounters four or more Ts in succession, and the resulting transcripts carry 3′-overhangs of one to four Us (the termination sequence). Such 3′-overhangs are similar to the 3′-overhangs described as advantageous for siRNAs.

A growing body of evidence demonstrates that delivery of small RNAs to nontargeted cells can be deleterious and/or lead to nonspecific effects. Thus, for practical applications, selective delivery of small RNAs to the targeted cell population would be advantageous. Since Pol III promoters are essentially ubiquitous, DNA based small RNA delivery methods are not transcription targeted and are thus can be subject to these nonspecific and toxic side effects. Several methods of targeting small RNAs have been explored including LoxCre, Tet, ligand-affinity mediated liposome encapsulated delivery etc. LoxCre and Tet are DNA based methods that rely of DNA regulatory regions placed 5′ and 3′ to the Pol III promoter. In some strategies, the DNA regulatory regions are placed within the Pol III promoter or replaces part of it. These methods can involve relatively large amounts of DNA (i.e. dicistronic as in the case of the LoxCre method where the Cre enzyme is under the expression control of a Pol II promoter and the Lox sites when excised bring the shRNA into register with the full U6 promoter), can rely on the exogenous addition of Tet or some other antibiotic or ligand, and can be “leaky” since they have the full active U6 promoter in place. Thus a clear simple method for creating targeted monocistronic small RNA promoters would be advantageous.

Pol II promoters display a wide range of endogenous targeting patterns. The expression profiles of Pol II promoters include (non inclusive) tissue specificity, tumor specificity, organ specificity, radiation specificity, ligand specificity (i.e. including estrogen, tamoxifen etc), ultrasound specificity, inflammation specificity, viral specificity, and various disease specificities. It is common practice to identify genes activated by specific conditions using gene-chip micro arrays and clone their promoters for use as pol II promoters activated by the specific condition. Thus, there is a very large number of promoters with known expression profiles and a systematic way to identify addition promoters with clinically or scientifically interesting expression profiles. No similar collection of pol III promoters with interesting expression profiles exists.

### SUMMARY OF THE INVENTION

The present invention is based, at least in part, on the discovery that constructs generated to include promoters with a basal region(s) of a Pol III promoter and a regulatory region(s) of a Pol II promoter can be used to target and/or regulate expression of short RNA molecules in cells. Such constructs take advantage of some of both Pol III and Pol II promoter functions, namely obtaining RNA with a specific length and obtaining RNA with a specific expression profile. Some currently available constructs include full Pol III promoters, such as U6 promoters, which do not allow for specific targeting and/or regulation. The present application features constructs with a specific expression profile, similar to a Pol II expression profile.

The present invention concerns genetic constructs that can be used to target and/or regulate expression of short RNA molecules in cells, particularly dsRNA molecules that can participate in RNA inhibition (RNAi), microRNA (miRNA) mediated expression regulation, such as siRNAs, short hairpin RNAs (shRNA), and miRNAs or RNA aptamers, e.g., riboswitches. Such regulation can be of many different types, such as spatial (e.g., in particular cells or tissues, including tumor-specific expression), temporal (occurring at particular times, such as particular development stages, and environmental (in response to particular environmental conditions), such as in response to radiation.

Generally, such genetic constructs include a sequence that will bind a RNA polymerase III complex, along with regulatory elements from a RNA polymerase II promoter region or regions. For example, such genetic constructs can be constructed as a fusion between a Pol III basal promoter region operatively linked with cis-acting regulatory region or regions (e.g., specific regulation enhancer and/or repressor elements) from a Pol II promoter region(s). Likewise, such genetic constructs can be constructed by mutagenizing a Pol II basal promoter region or regions such that it binds a Pol III complex. Such a mutant or modified sequence can be constructed by various methods, such as by mutation of a parent sequence or by chemical synthesis. However produced, the present genetic constructs that provide Pol III binding along with Pol II regulatory elements are referred to herein as “fusion promoters”, or alternatively as “chimeric promoters”.

Such fusion promoters can be used to provide regulated expression of inhibitory RNA molecules for the various applications of such inhibitory RNA molecules, generally involving gene knock-down or knock-out. For example, such uses include gene function analyses, drug development, gene pathway studies, development of RNA-based therapeutics, therapeutic and prophylactic applications, and as controls or indicators in small molecule drug screening and development.

In one aspect, the disclosure features a nucleic acid construct that includes a Pol III/Pol II fusion promoter. The fusion promoter includes an RNA Polymerase III-binding basal promoter region, one or more cis-acting regulatory regions from a Pol II promoter operably linked with that basal promoter region. The cis-acting regulatory region or regions provide specific regulation of expression from the construct. In particular embodiments, a nucleic acid construct includes two linked Pol III/Pol II fusion promoters having different specific regulation characteristics.

In certain embodiments, the cis-acting regulatory region or regions provide cell-specific regulation; tissue-specific regulation; cell-cycle specific regulation; tumor-specific regulation in vivo; radiation-induced expression in vivo; estrogen-induced expression in vivo; ligand-induced expression in vivo; pattern specific expression in vivo such as expression in the same distribution as a virus or the same distribution as the expression of a viral gene or expression in the distribution similar to an RNA polymerase type II promoter like developmental program or immune specific or regional specific in vivo; ultrasound induced expression in vivo; heat induced expression in vivo; cold induced expression in vivo (e.g., metallothionein-1); glucose induced expression in vivo; hyperglycemic induced expression in vivo; disease induced expression in vivo; inflammation induced expression in vivo (e.g., acid-sensing ion channel (ASIC) polypeptides such as ASIC3 and mucosal addressin cell adhesion molecule-1 (MAdCAM-1) such as in inflammatory bowel disease (IBD), cyclooxygenase-2 (COX-2) such as in human pulmonary epithelial cells); tissue response induced expression in vivo; light induced expression in vivo (e.g., fos, NGFI-A, and NGFI-B); medication induced expression in vivo; apoptosis induced expression in vivo; spreading depression induced expression in vivo (e.g., atrial natriuretic peptide, COX-2, TNF-alpha, IL-1beta, galanin, and metalloproteinases such as MMP-9); infarction induced expression in vivo (e.g., P-selectin); pulmonary embolism induced expression in vivo; hypoxia induced expression in vivo (e.g., hypoxia inducible factor 1 alpha, vascular endothelial growth factor (VEGF), endothelial growth response 1 (Egr-1), erythropoietin); stroke induced expression in vivo; and combinations thereof.

In certain embodiments, the construct also includes a sequence encoding an RNAi agent operably linked with the fusion promoter, e.g., a shRNA or siRNA (i.e., encoding the two strands of an siRNA). In particular embodiments, the RNAi agent is targeted to mRNA of a gene associated with a disease or condition. A variety of such genes have been identified; some of which have been targeted and inhibited using RNAi.

In particular embodiments, the basal promoter region is from a Pol III promoter (e.g., a U6 basal promoter, an H1 basal promoter, a tRNA basal promoter); has the sequence of a Pol III basal promoter; is a mutated Pol II basal promoter that preferentially binds Pol III instead of Pol II.

In particular embodiments, the cis-acting regulatory region or regions include the entire regulatory region from a Pol II-transcribed gene, except for the basal promoter elements. In particular embodiments, the cis-acting regulatory region or regions include CMV early intermediate regulatory region or regions.

In another aspect, the invention also provides a vector that includes a Pol III/Pol II fusion promoter of the present invention, e.g., as described above or otherwise described herein.

In particular embodiments, the vector is a plasmid, a viral-based vector; a cosmid; a YAC, or a BAC. In particular embodiments, the vector is replication defective; the vector is replication competent.

Similarly, in another related aspect, the invention concerns a cell that includes a Pol III/Pol II fusion promoter of the invention operably linked with a coding sequence, such as an RNAi agent such as an shRNA or siRNA. The fusion promoter and linked RNAi agent-encoding sequence can be in a vector as described herein or incorporated in a chromosome(s).

In certain embodiments, the cell is in cell culture; is in an animal, e.g., a human, a feline, a canine, a bovine, a porcine, an ovine, an equine animal, a bird; a fungus; a plant. In particular cases, the cell is an animal cell, e.g., a human cell, a feline cell, a canine cell, a bovine cell, a porcine cell, an ovine cell, an equine cell; a bird cell; an insect cell; a plant cell.

In yet another related aspect, the invention provides a non-human transgenic organism that includes a plurality of cells that include a genetic construct of the present invention.

In particular embodiments, such cells are as described above or otherwise herein; the organism is as described herein.

Likewise, in another aspect, the invention provides a kit that includes a packaged amount of one or more genetic constructs of the present invention. Typically such a kit also includes additional component(s), such as instructions for use; the genetic construct is packaged in single use form; the genetic construct is in a vector; the genetic construct also includes a coding sequence operably linked with the Pol II/Pol II fusion promoter; the genetic construct is formulated in a pharmaceutical composition; the kit also includes a second active compound; the genetic construct is packaged in unit dose form.

Another aspect of the invention concerns a pharmaceutical composition that includes a genetic construct of the invention, where the genetic construct also includes an RNAi agents such as an shRNA or siRNA sequence operatively linked with the fusion promoter, and a pharmaceutically acceptable carrier or excipient.

In certain embodiments, such pharmaceutical composition is formulated as an injectable composition; formulated for topical administration; formulated as a liposomal composition; includes a vector containing the construct; includes a viral vector that includes the construct; includes a plurality of vectors containing different constructs.

A further aspect concerns a method for making a genetic construct, of the present invention by operably linking a nucleic acid sequence encoding an RNAi agent with a Pol III/Pol II fusion promoter of the invention.

In certain embodiments the construct, operably linked coding sequence, specific regulation properties, and/or other characteristics of the construct or its use are as described herein, e.g., RNAi agent is an shRNA, or siRNA.

In connection with the use of the present constructs and related materials, another aspect of the invention concerns a method for expressing an RNAi agent in a cell by maintaining a cell under expression conditions, where the cell includes a genetic construct of the present invention operably linked with a RNAi agent encoding sequence. In some embodiments RNAi agent is shRNA; siRNA.

Likewise, another aspect concerns a method for inhibiting expression of a target gene in a cell. The method involves transfecting the cell with a vector that includes a genetic construct of the present invention operably linked with a nucleic acid sequence encoding an RNAi agent targeted to the target gene, and maintaining the cell under expression conditions.

In particular embodiments, the cell is in a organism (e.g., as described herein); the construct includes a tissue-specific regulatory element and the target gene is preferentially inhibited in cells of tissue corresponding to that tissue-specific regulatory element; the construct includes a tumor-specific regulatory element and the target gene is preferentially inhibited in cells of tumors corresponding to that tumor-specific regulatory element; the inhibition is induced in response to radiation; the inhibition is induced in response to the presence of an effective amount of a non-peptide and non-nucleotidic chemical species, e.g., an estrogen.

Further, another aspect concerns a method for analyzing gene function, which involves inhibiting expression of a gene in a cell, where the inhibiting is due to expression of an RNAi agent from a genetic construct of the present invention operably linked with a nucleic acid sequence encoding the RNAi agent; determining a biological change in the cell following the inhibiting, where such biological change is indicative of the function of the gene.

In particular embodiments, the determining involves comparing at least one biological characteristic with a control cell in which expression of the gene is not inhibited; the method also involves transfecting the cell with a vector that includes the genetic construct, e.g., a viral vector of a plasmid.

Another aspect provides a method for validating a target as a therapeutic target, and includes inhibiting expression of a putative therapeutic target gene in the cell, where the inhibiting is due to expression of an RNAi agent from a genetic construct of the invention operably linked with a nucleic acid sequence encoding the RNAi agent, and determining whether a biological change in the cell following that inhibiting corresponds with a therapeutic effect. Correspondence of the biological change with the therapeutic effect is indicative that the gene is a therapeutic target gene.

In another aspect, the invention provides a useful test control, thus providing a method for positive control of a biological effect of a small molecule test compound. The method involves contacting a first cell with a test compound; inhibiting a target gene in a comparison cell using expression of an RNAi agent from a genetic construct of the present invention operably linked with a sequence encoding the RNAi agent, and comparing the effect of the test compound in the first cell with the effect of inhibition of the target gene is the comparison cell.

In particular embodiments, the test compound is pre-selected to be active on the target gene; comparing includes determining whether the test compound has effects additional to the effects of the inhibiting by the RNAi agent.

In still another aspect, the invention provides a method for treating a disease or condition in which inhibition of a target gene provides a beneficial effect. The method includes administering a pharmacologically effective amount of a nucleic acid construct, vector, cell, kit, or a pharmaceutical composition that includes a genetic construct of the invention operably linked with a sequence encoding an RNAi agent targeted to the target gene, to a subject suffering from or at risk of such disease or condition. The disease or condition can be, e.g., a cancer, an infectious disease, or a neurodegenerative disease, e.g., caused by mutations in SOD1 gene.

In particular embodiments, the vector is a plasmid; the vector is a viral vector; the subject is a human; the subject is a non-human animal; the subject is a plant; RNAi agent is shRNA; RNAi agent is siRNA.

Also within the invention is the use of disclosed nucleic acid constructs, vectors, cells, kits, or pharmaceutical compositions in the treatment or prevention of a disease or condition wherein inhibition of a target gene provides a beneficial effect. The disease can be, e.g., a cancer, an infectious disease, or a neurodegenerative disease, e.g., one caused by mutations in SOD1 gene. In one aspect, the disclosure features use of a vector including a genetic construct comprising a Pol III/Pol II fusion promoter providing specific regulation of expression, operably linked with a sequence encoding an RNAi agent, wherein said fusion promoter comprises a RNA Polymerase III-binding basal promoter region and cis-regulatory region or regions from a Pol II promoter operably linked with said basal promoter region, wherein said cis-acting regulatory region or regions provide specific regulation of expression from said fusion promoter for treatment of a disease or condition wherein inhibition of a target gene provides beneficial effect.

In particular embodiments, the vector is a plasmid; the vector is a viral vector; the RNAi agent is shRNA; the RNAi agent is siRNA.

Also within the invention is the use of disclosed nucleic acid constructs, vectors, cells, kits, or pharmaceutical compositions in the manufacture of a medicament for treatment or prevention of a disease or condition wherein inhibition of a target gene provides a beneficial effect. The medicament can be in any form described herein. The disease can be, e.g., a cancer, an infectious disease, or a neurodegenerative disease, e.g., one caused by a mutation or mutations in SOD1 gene. In one aspect, the disclosure features use of a vector including a genetic construct comprising a Pol III/Pol II fusion promoter providing specific regulation of expression, operably linked with a sequence encoding an RNAi agent, wherein said fusion promoter comprises a RNA Polymerase III-binding basal promoter region and cis-regulatory region or regions from a Pol II promoter operably linked with said basal promoter region, wherein said cis-acting regulatory region or regions provide specific regulation of expression from said fusion promoter in preparation of a medicament for treatment of a disease or condition wherein inhibition of a target gene provides a beneficial effect.

In particular embodiments the vector is a plasmid; the vector is a viral vector; the RNAi agent is shRNA; the RNAi agent is siRNA.

As used in connection with the present constructs, the term “cis-acting regulatory region” or “regions” refers to nucleic acid sequences in the vicinity of a structural gene portion that affects the transcription of the structural gene.

As used in connection with nucleotide sequences, the term “encodes” indicates that the nucleotide sequence or molecule (generally DNA) contains a sequence that is complementary to a reference RNA sequence. Thus, a DNA sequence that encodes a particular RNA molecule can produce such RNA molecule when operatively linked with suitable control sequences and in the presence of necessary reaction components. In reference to amino acid sequences, the term “encodes” means that the indicated nucleotide sequence has a sequence that can be translated to the indicated amino acid sequence (in the case of an RNA sequence) or transcribed to a complementary RNA which can be translated to the indicated amino acid sequence (in the case of a DNA sequence) when such nucleotide sequences are operatively linked with suitable control sequences and in the presence of necessary reaction components.

As used herein, the terms “genetic construct” and “construct” refer to genetically engineered DNA molecules that include a basal promoter operatively linked with one or more enhancer and/or repressor regulatory regions. The construct can also include additional sequences, such as a shRNA coding region operatively linked with the basal promoter and enhancer and/or repressor regulatory regions.

The term “enhancer” refers to a DNA sequence which, when bound by a specific protein factor(s), enhances the level of expression of a gene, but is not sufficient alone to cause expression. In many cases, an “enhancer” is capable of enhancing expression of a gene even if located a substantial distance from the gene and in either sequence orientation relative to the gene.

In connection with the present invention, the term “kit” refers to a packaged manufacture (e.g. in a box, bottle, vial, or other container or combination of containers) that includes at least one reagent, e.g. a construct, for activating RNAi in a cell or organism. In particular embodiments, the kit is prepared containing one or more unit dose preparations of the present constructs.

In connection with the present genetic constructs, the term “unit dose” refers to a quantity of the construct designed and suitable for single use, e.g., for a single therapeutic administration or a single knock-down test for a gene.

The term “intron” refers to a sequence within the coding sequences of a gene that is not translated into protein. Such intron is transcribed into RNA but is removed (by RNA splicing) before the RNA is translated into protein.

The term “gene” includes genomic DNAs, cDNAs, RNA, or other polynucleotides that encode gene products, and includes introns and control sequences that affect transcription, translation, or other regulation and/or processing function.

The terms “exogenous gene” and “foreign gene” refer to a gene that has been obtained from an organism or cell type other than the organism or cell type in which it is expressed. Unless expressly indicated to the contrary; these terms also include a gene from the same organism that has been translocated from its normal situs in the genome. Similarly, the terms “exogenous sequence”, “foreign sequence” and the like refer to nucleotide sequences from such other source or location.

As used herein the term “target gene” refers to a gene intended for downregulation (i.e., inhibition), such as by using RNA interference (“RNAi”). Similarly, the term “target RNA” refers to an RNA molecule, e.g., a mRNA molecule intended for downregulation (e.g., via RNAi-induced degradation).

As used herein in connection with the present nucleic acid constructs, the term “promoter” refers to a DNA sequence to which RNA polymerase can bind and initiate transcription of an operably linked coding sequence, along with associated regulatory elements that provide additional transcriptional control (e.g., binding elements for other transcription factors).

The term “Pol III promoter” refers to an RNA polymerase III promoter. Examples of Pol III promoters include, but are not limited to, the U6 promoter, the H1 promoter, and the tRNA promoters.

By “Pol II promoter” is meant an RNA polymerase II promoter. Examples of Pol II promoters include, but are not limited to, the Ubiquitin C promoter and the CMV early intermediate promoter.

In the context of the production a product from a gene or coding region, the term “expression” refers to the enzymatic synthesis of the product via transcription and/or translation processes, and includes expression in a cell(s) as well as transcription and/or translation of nucleic acid(s) in cell-free expression systems, cloning systems, and the like.

As used herein, the terms “RNA interference” and “RNAi” refer to a sequence-specific process by which a target molecule (e.g., a target gene, protein or RNA) is downregulated via downregulation of expression. Without being bound to a specific mechanism, as currently understood by those of skill in the art, RNAi involves degradation of RNA molecules, e.g., mRNA molecules within a cell, catalyzed by an enzymatic, RNA-induced silencing complex (RISC). RNAi occurs in cells naturally to remove foreign RNAs (e.g., viral RNAs) triggered by dsRNA fragments cleaved from longer dsRNA which direct the degradative mechanism to other RNA sequences having closely homologous sequences. As practiced as a technology, RNAi can be initiated by human intervention to reduce or even silence the expression of target genes using either exogenously synthesized dsRNA or dsRNA transcribed in the cell (e.g., synthesized as a sequence that forms a short hairpin structure).

As used herein, the term “RNAi agent” refers to an RNA (or RNA analog) that includes a sequence having sufficient sequence complementarity to a target RNA to direct RNAi to the target RNA. Such sequence complementarity may be complete complementarity, but may include a low level of mismatches, e.g., 3′ or 5′ terminal mismatches.

The term “RNA”, “RNA molecule”, and “ribonucleic acid molecule” refer to a polymer of ribonucleotides. Unless expressly indicated to the contrary, such ribonucleotides includes ribonucleotide analogs. Similarly, the terms “DNA”, “DNA molecule”, and “deoxyribonucleic acid molecule” refer to a polymer of deoxyribonucleotides. Unless expressly indicated to the contrary, such deoxyribonucleotides include deoxyribonucleotide analogs. DNA and RNA can be synthesized using enzymatic replication or transcription mechanisms (e.g., in a cell or in a cell-free enzymatic synthetic system), or can be chemically synthesized. RNA, in particular, can be post-transcriptionally modified on one or more ribonucleotides. DNA and RNA can be single-stranded (i.e., ssDNA and ssRNA) or multi-stranded, which is most commonly double stranded (i.e., dsRNA and dsDNA).

In the context of RNAi, the term “sequence-specific” means sufficient sequence complementarity to a target RNA molecule sequence to preferentially direct RNAi-induced degradation of such molecule. It does not mean that the RNAi agent is perfectly complementary to the target sequence or that there is no off target degradation directed by the agent.

The terms “mRNA” and “messenger RNA” are used conventionally to refer to a single-stranded RNA that has a sequence that encodes the amino acid sequence(s) of one or more polypeptide chains. Such coding sequence is translated during protein synthesis, producing the corresponding amino acid sequence.

The term “transcript” refers to a RNA molecule transcribed from a DNA or RNA template by a RNA polymerase. The term “transcript” includes RNAs that encode polypeptides (i.e., mRNAs) as well as noncoding RNAs (“ncRNAs”).

As used herein, the terms “small interfering RNA” and “short interfering RNA” (“siRNA”) refer to a short RNA molecule, generally a double-stranded RNA molecule about 10-50 nucleotides in length (the term “nucleotides” including nucleotide analogs), preferably between about 15-25 nucleotides in length. In most cases, the siRNA is 17, 18, 19, 20, 21, 22, 23, 24, or 25 nucleotides in length. Such siRNA can have overhanging ends (e.g., 3′-overhangs of 1, 2, or 3 nucleotides (or nucleotide analogs). Such siRNA can mediate RNA interference.

As used in connection with the present invention, the term “shRNA” refers to an RNA molecule having a stem-loop structure. The stem-loop structure includes two mutually complementary sequences, where the respective orientations and the degree of complementarity allow base pairing between the two sequences. The mutually complementary sequences are linked by a loop region, the loop resulting from a lack of base pairing between nucleotides (or nucleotide analogs) within the loop region.

The term “subject” refers to a living higher organism, such as an animal (e.g., a mammal or a bird) or a plant. Examples of animal subjects include humans, monkeys, cows, horses, sheep, goats, dogs, cats, mice, rats, and transgenic derivatives or variants thereof. The term “treatment”, as used herein, means the application or administration of a therapeutic agent to a subject (or application or administration of a therapeutic agent to an isolated tissue or cell line from a subject) who has a disease or condition, a symptom of a disease or condition, a predisposition toward a disease or condition, or is otherwise at risk of contracting the disease or condition. Such treatment is intended to relieve at least in part at least one symptom of the disease or condition, to alter the course of the disease or condition, and/or to reduce the likelihood that the subject will develop the disease or condition, e.g., to cure, heal, alleviate, relieve, alter, remedy, ameliorate, improve, or affect the disease or condition, the symptoms of the disease or condition, the predisposition toward a disease or condition, or the likelihood of developing the disease or condition.

As used herein, the term “therapeutic agent” means a composition, e.g., a molecule that produces a therapeutic effect when administered or applied to a subject suffering from or at risk of a disease or condition. Such therapeutic agents can, for example, be small molecules, peptides, antibodies, ribozymes, antisense oligonucleotides, chemotherapeutic agents, and radiation.

The term “effective amount”, as used here in, is defined as that amount sufficient to produce a particular pharmacological effect.

The term “therapeutic amount” refers to an amount sufficient to treat or prevent a particular disease or condition. Such amount can vary depending on such factors as the size, weight, and condition of the subject, the type of the disease or condition, the particular agent being administered, and the method and route of administration of the agent. One of ordinary skill in the art determine such therapeutic amount of the agent without undue experimentation.

The term “mutation” refers to a substitution, addition, or deletion of a nucleotide or small number of nucleotides within a gene sequence. Such mutations can result in aberrant production (e.g., misregulated production) of the protein encoded by the gene sequence, production of an aberrant or variant product, or can be silent.

The term “nucleoside” refers to a molecule having a purine or pyrimidine base covalently linked to a ribose or deoxyribose sugar. Exemplary nucleosides include adenosine, guanosine, cytidine, uridine and thymidine. The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The term “pharmaceutical composition” as used herein, refers to an active agent formulated with one or more compatible fillers, diluents, carriers, excipients, or encapsulating substances which are suitable for administration to a human or other animal subject.

Certain methods of the instant invention include comparing a value, level, feature, characteristic, property, etc. to a “suitable control” (also referred to as an “appropriate control”). Such a control is any control or standard acceptable to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing an RNAi agent of the invention into a cell or organism or in a reference cell or organism.

The term “upstream” refers to nucleotide sequences that precede, e.g., are on the 5′ side of, a reference sequence.

The term “downstream” refers to nucleotide sequences that follow, e.g., are on the 3′ side of, a reference sequence.

As used in connection with the present invention, the term “vector” refers to a nucleic acid molecule capable of transporting another nucleic acid to which it has been linked into a cell. Such vectors include plasmids, viral vectors, cosmids, YACs, BACs, and the like. “Plasmids” are small circular double stranded DNA molecules which replicate independently of the cellular genome. Typically such plasmids include one or more sites into which additional DNA segments can be inserted and ligated. “Viral vectors”, which are vectors based on viral genomes, which may be engineered and/or recombinant viral vectors. Often, such viral vectors have non-essential genes removed, may be engineered to add cloning sites, and/or may be selected or modified to be an attenuated virus and/or to be replication defective or to have other selected properties. Examples of viral vectors include vectors derived lentiviral (e.g., HIV, SIV, EAIV, FIV), adenovirus, adeno-associated virus, oncoretrovirus, pox virus (e.g., vaccinia virus and caarypox virus), herpesvirus, foamyvirus, MMLV virus (Moloney murine leukemia virus), baculovirus, alphavirus (e.g., Semliki Forest virus (SFV), Sindbis virus (SIN), and Venezuelan Equine Encephalitis virus (VEE). Three different types of alphavirus vectors have been constructed. I Replication-deficient vectors: RNA molecules containing the viral nonstructural genes (nsP1-4) and the foreign gene of interest are packaged into alphavirus particles with the aid of a helper vector containing the viral structural genes. The generated recombinant alphavirus particles are capable of infection of host cells, but because no viral structural genes are accommodated, no further virus replication occurs. The obtained transgene expression is therefore of a transient nature. II Replication-competent vectors: In contrast to the suicide vectors described above, these vectors contain a second subgenomic promoter and the foreign gene of interest added to the full-length alphavirus genome. Infection of host cells with replication-competent particles will obviously lead to virus replication. III Layered DNA-vectors: An RNA polymerase II expression cassette is introduced to drive the transcription of a self-amplifying RNA (replicon) vector, which allows direct use of plasmid DNA for transfection and expression studies (Berglund et al., 1996, Dubensky et al., 1996).), and parvovirus vectors.

Additional embodiments will be apparent from the Detailed Description and from the claims.

### BRIEF DESCRIPTION OF THE DRAWINGS

FIG. 1 schematically shows the design of Pol III/Pol II fusion promoters utilizing U6 basal promoter with GFAP regulatory elements. Two chimeric promoters where created: 1) A chimeric promoter that included the A and B cis-acting elements of the human GFAPp element linked to the core promoter of U6 termed GFAP-EcoNI which was cloned into a plasmid termed pGFAP-EcoNI (or pGFAP-EcoNI-Control) and 2) A chimeric promoter that included the A, B, and D regions of the human GFAPp element linked to the core promoter of U6 termed GFAP-SmaI and was cloned into a plasmid termed pGFAP-SmaI (or pGFAP-SmaI-Control). Two additional plasmids were created that contained the respective chimeric promoters driving the expression of shRNAs directed against eGFAP termed pGFAP-EcoNI-eGFP and pGFAP-SmaI-eGFP. These four plasmids were used to demonstrate shRNA expression from the chimeric promoters and examine the transcript expression program of the chimeric promoters.

### DETAILED DESCRIPTION OF THE PREFERRED EMBODIMENTS

### I. General

Double-stranded RNA (dsRNA)-induced sequence-specific gene silencing is known as RNA interference (RNAi). There is a growing appreciation of the vast therapeutic potential of RNAi for treating a wide range of diseases including cancers and infectious diseases (Bi et al. Curr Gene Ther 2003, 3(5):411-417; Brisibe et al. Trends Biotechnol 2003, 21(7):306-311; Caplen Expert Opin Biol Ther 2003, 3(4):575-586; Lieberman et al. Trends Mol Med 2003, 9(9):397-403; Wang et al. World J Gastroenterol 2003, 9(8):1657-1661; Wolff & Herweijer Ernst Schering Res Found Workshop 2003(43):41-59). In addition, RNAi is a powerful tool for basic scientists to explore the functions of genes through reverse genetic manipulations (Scherr et al. Curr Med Chem 2003, 10(3):245-256; Szweykowska-Kulinska et al. Acta Biochim Pol 2003, 50(1):217-229; Wimmer Nat Rev Genet 2003, 4(3):225-232). As the role of RNAi in both science and medicine continues to grow, targeted delivery of short dsRNAs (siRNA) to the desired tissue will become increasingly important. Some reports have indicated that chemically synthesized siRNA can be targeted using physical targeting technologies like nanogels and PEGylated immunoliposomes. DNA-based RNAi approaches offer the advantage of being both less labile than unmodified dsRNA and displaying amplification, i.e. many dsRNAs can be expressed from a single delivered DNA promoter construct. Described approaches for targeting DNA-based RNAi constructs include Lox/Cre based approaches and physical targeting of the DNA constructs using nanogels or PEGylated immunoliposomes. However, the present simple transcriptional targeting method for delivery of siRNA represents an important advance in RNAi therapy.

In the DNA-based approach to RNAi, DNA based constructs are used to express short dsRNAs in cells in the form of either short hairpin RNA (shRNA) or expression of both strands of the double stranded complementary sequences. Each of these approaches has typically relied on the use of an RNA polymerase type III promoter so that transcription is terminated at the appropriate length. While some uses of RNA polymerase type II promoters have been described, generally an RNA polymerase promoter type II yields much longer RNA molecules which has been shown to activate cellular inflammatory cascades and other non-specific effects.

Both RNA polymerase type II promoters and RNA polymerase type III promoters have a structure that includes a core (or basal) promoter and a collection of enhancers, silencers and other elements. Typically, the respective holoenzyme attaches to the core promoter region along with a collection of transcription factors to create the preinitiation complex. The transcription rate is then largely controlled by the action of enhancers, silencers and other elements located both 5′ and 3′ to the core promoter. This basic mechanism is responsible for the complex transcriptional targeting displayed by RNA polymerase type II promoters including (non-exhaustive) tissue specific, tumor specific, radiation inducible, estrogen inducible, cell-cycle dependent and organism specific promoters.

While RNA polymerase III promoters have the advantage of appropriate termination, currently characterized RNA polymerase type III promoters display nearly ubiquitous expression. In contrast to RNA Pol III promoters, RNA polymerase type II promoters display a rich array of transcriptional control, but are still not generally appropriate for use for expression of siRNAs or shRNAs due to the production of long RNA molecules.

Thus, the present invention concerns the use enhancers, silencers and other regulatory elements from an RNA polymerase type II promoter to regulate the transcription rate of an RNA polymerase type III preinitiation complex to create a chimeric RNAi expression promoter. Changing an RNA polymerase type II promoter such that an RNA polymerase type III would form a transcriptional complex instead of a polymerase type II yields an RNAi promoter with expression characteristics similar to the parent RNA polymerase type II promoter while retaining the polymerization and termination properties characteristics of RNA Pol III.

There are several potential ways to accomplish this, including exchanging the core promoter of an RNA polymerase type II promoter with one from an RNA polymerase type II promoter, and creating mutations to the core promoter region for an RNA polymerase II promoter such that it preferentially binds a RNA Polymerase III. Further, different combinations of enhancers, silencers and other elements from both promoters may be combined to achieve an RNAi promoter with desired expression characteristics.

Thus, the present invention concerns specifically regulatable genetic constructs for the expression of RNAs, in particular RNA agents for activating RNAi such as shRNAs and siRNAs. Such regulation can be spatial, temporal, or environmental. The present regulatable genetic constructs include fusion promoters that include a basal promoter that binds an RNA polymerase III, operably linked with at least one additional regulatory element from a RNA polymerase II promoter region.

Surprisingly, such fusion promoters offer the advantageous synthetic properties associated with Type III polymerases, while also offering the regulatory range and flexibility of Type II polymerase regulation.

Thus, the present invention provides compositions for RNA interference and methods for preparing and using such compositions. The compositions are useful for the range of applications of RNAi, including determining and analyzing gene functions for both normal and mutant genes, determining and analyzing gene pathways, analyzing and validating putative drug targets, and targeting genes for therapeutic and prophylactic applications. Advantageously, such applications can be carried out in a regulated manner, with a variety of different regulatory characteristics available for use.

### II. Fusion Promoters

A. Design and Construction of Pol III/Pol II Fusion (Chimeric) Promoters

A variety of methods can be used to produce the present fusion promoters using standard techniques. On such approach is to create a fusion promoters is to replace a RNA Pol II basal promoter with a RNA Pol III basal promoter (e.g., using conventional cloning techniques). The resulting construct has the regulatory elements from a Pol II regulatory region associated with a Pol III basal promoter. The converse can also be performed, with one or more regulatory elements from a Pol II regulatory region linked with a Pol III basal promoter. In either case, the result is a chimeric nucleic acid, with a Pol III basal promoter and at least one additional regulatory element from a Pol II regulatory region.

For example, the U6 promoter, an RNA polymerase type III promoter, has a basal or core promoter and two regulatory elements termed the PSE and the DSE. The glial acid fibrillary protein (GFAP) protein promoter, a glial cell selective promoter, has a core promoter and three regulatory regions 5′ to the core promoter and one 3′ to the promoter. It is possible to create a chimeric promoter consisting of the basal promoter of the U6 and the regulatory regions of the GFAP promoter that displays tissue selective expression of RNAi. This evidence supports the broader concept that this methodology could be used to systematically create a broad range of RNAi promoters with interesting expression targeting characteristics. These include, but are not limited to, creation of other tissue specific promoters, radiation inducible promoters, ligand inducible promoters, estrogen inducible promoters, organism specific promoters (i.e. viral specific promoters that express in the same general distribution of the replication of the virus or parasite or yeast specific promoters), tumor specific promoters, cell-cycle dependent promoters and developmental stage specific promoters.

B. Mutated Promoters

It has been demonstrated that simple point mutations to the TATA box region can convert a promoter from an RNA polymerase type II promoter into an RNA polymerase type III promoter. This approach can be used to convert RNA polymerase type II promoters into RNAi promoters while retaining the targeting characteristics of the RNA polymerase type II promoter. For example, mutation of the TATA box of glial fibrillary acid protein (GFAP) from ATAA to AATAT converts it into an RNAi promoter with tissue selective expression.

Similarly, a wide range of RNA polymerase type II promoters can be converted into RNA polymerase type III promoters using this simple methodology. This results in the creation of targeting RNAi promoters with a variety of characteristics including, but not be limited to, creation of other tissue specific promoters, radiation inducible promoters, ligand inducible promoters, estrogen inducible promoters, organism specific promoters (i.e. viral specific promoters that express in the same general distribution of the replication of the virus or parasite or yeast specific promoters), tumor specific promoters, cell-cycle dependent promoters and developmental stage specific promoters.

C. More General Mixing of Regulatory Elements and Combination of Directed Mutagenesis and Mixing of Regulatory Elements to Create RNAi Promoters.

Creation of RNAi promoters can include the mixing of multiple types of regulatory elements (in addition to the mixing of core promoters or in lieu of mixing of core promoters) or a combination of directed mutagenesis with any degree of combination of mixing of regulatory elements. In addition, compound RNAi promoters with regulatory elements from multiple types of RNA polymerase type II promoters may be used to produce more specific control of RNAi expression. This would include, but is not limited to, using regulatory elements from two promoters with similar expression characteristics like two promoters that are glial specific to improve the targeting of RNAi expression to glial cells or using regulatory elements from a tissue specific RNAi promoter with elements from a tumor specific RNAi promoter to create a chimeric promoter capable of targeting tumors in particular tissues. Including or leaving out specific regulatory elements can be used to control the degree of targeting or even expand or completely change the targeting of the RNAi promoter.

D. Pol III Promoters

Many different Pol III promoters can be used in construction of the present Pol III/Pol II fusion promoters. Well-known examples of promoters include the U6 promoter, the H1 promoter, and tRNA promoters (e.g., selenocysteine tRNA gene (TRSP)). The sequences of those promoters are known and can be manipulated by conventional molecular biology methods to create recombinant nucleic acid constructs.

Other Pol III promoters include the 7SL RNA promoter (e.g., Arabidopsis, human, or mouse), and the RNase P RNA (RPPH1) gene promoter (e.g., from the domestic dog (Canis familiaris)), and the adenoviral VA1 polymerase III (pol III) promoter. Additional Pol III promoters that are known or identified can also be used. Such promoters can be identified by methods similar to the methods by which prior Pol III promoters have been identified.

E. Pol II Regulatory Region Elements

Similarly to Pol III promoters, many different Pol II regulatory regions and elements are known and more are being identified. Such regions and elements can be used to construct the present fusion promoters. Pol II promoters and their associated regulatory elements are notable for the variety of specific regulation demonstrated for the corresponding genes. Such regulatory elements can be incorporated in the present Pol III/Pol II fusion promoters, thereby providing specific regulation of expression from the fusion promoter of an operably linked coding sequence.

Examples of the specific regulation provided by such Pol II regulatory elements include cell type specific, tissue specific, cell cycle specific, development stage specific, radiation induced, and hormone induced regulation. Elements providing different types of regulation can even be used in combination to provide multiple types of regulation with a single construct and/or additive or synergistic specific regulatory effects.

### III. Nucleic Acids Encoding RNAi Agents and Other RNA Molecules

The present nucleic acid constructs include those with a fusion promoter with an operably linked nucleic acid sequence encoding an RNAi agent or other short RNA such as micro RNA (miRNA). Such RNAi agents include siRNAs and shRNAs, as well as longer sequences that are processed by RNAi machinery to shorter sequences intracellularly.

A shRNA-encoding nucleic acid sequence or molecule includes a first sequence (or portion) and a second sequence (or portion) that have nucleotide sequences such that the RNA sequences encoded by those portions are sufficiently complementary to hybridize with each other to form a duplex or double-stranded stem portion. Such sufficient complementarity does not require that the portions are fully or perfectly complementary. The stem-forming portions are connected by a portion (referred to as a loop-portion or loop-encoding portion) having a sequence such that the encoded RNA from that portion does not anneal or hybridize to other portions of the shRNA (i.e., forms a single strand loop). Such shRNA-encoding nucleic acid sequences or molecules are transcribed, thereby forming shRNAs. shRNAs can also include one or more bulges, i.e., extra nucleotides that create a small nucleotide “loop” in a portion of the stem, for example a one-, two- or three-nucleotide loop. The encoded stem portions can be the same length, or one portion can include an overhang of, for example, 1-5 nucleotides (e.g., a 3-overhang).

For shRNA, one strand of the stem portion of the encoded shRNA is sufficiently complementary (e.g., antisense) to a target RNA (e.g., mRNA) sequence to mediate degradation or cleavage of that target RNA via RNA interference (RNAi). The antisense portion can be on the 5′ or 3′ end of the stem. The stem-encoding portions of a shRNA-encoding nucleic acid (or stem portion of a shRNA) are typically about 15 to about 50 nucleotides in length. When used in mammalian cells, the length of the stem portions can be selected to be less than about 30 nucleotides to avoid provoking non-specific responses like the interferon pathway. In non-mammalian cells, the stem can be longer than 30 nucleotides. In fact, a stem portion can include much larger sections complementary to the target mRNA (up to, and including the entire mRNA). The loop portion in the shRNA (or loop-encoding portion in the encoding DNA) can be of various lengths, e.g., about 2 to about 20 nucleotides in length, i.e., about 2, 3, 4, 5, 6, 7, 8, 9, 10, 11, 12, 13, 14, 15, 16, 17, 18, 19, 20 or more nucleotides in length. Certain loop portions are or include a 4 nucleotide sequence, referred to as a “tetraloop” sequence. Without limitation, such tetraloop sequences include the sequences GNRA, where N is any nucleotide and R is a purine nucleotide, GGGG, and UUUU.

For siRNAs, the construct can be designed such that expression is from a bicistronic sequence, with inverted regions for the sense and antisense strands of the double stranded siRNA. The two complementary strands can then hybridize in the cell. Alternatively, expression of each strand can be driven from separate fusion promoters, which may be the same or different. In the case of different promoters, the promoters may be selected such that together they increase the specificity of regulation of dsRNA, e.g., a cell type specific promoter combined with a tumor specific promoter.

The sequence of the antisense portion of a siRNA or shRNA can be designed by selecting an 18, 19, 20, 21, 22, 23, 24, 25 nucleotide, or longer, sequence from within the target RNA (e.g., mRNA), for example, from a region 100 to 200 or 300 nucleotides upstream or downstream of the start of translation. In general, the sequence can be selected from any portion of the target RNA (e.g., mRNA) including the 5′ UTR (untranslated region), coding sequence, or 3′ UTR. This sequence can optionally follow immediately after a region of the target gene containing two adjacent AA nucleotides. The last two nucleotides of the nucleotide sequence can be selected to be UU. shRNAs and longer dsRNAs so generated are processed under appropriate conditions (e.g., in an appropriate in vitro reaction or in a cell) by RNAi machinery (i.e., Dicer and/or RISC complexes) to generate siRNAs. Single stranded RNAs (including shRNAs and miRNAs) can be synthesized exogenously or can be transcribed in vivo from an RNA polymerase (e.g., a Pol II or Pol III polymerase).

### IV. Vectors and Host Cells

The invention also concerns vectors that include the present constructs. Of particular benefit are expression vectors, especially those for expression in eukaryotic cells. Such vectors can, for example, be viral, plasmid, cosmid, or artificial chromosome (e.g., yeast artificial chromosome) vectors.

Typically, plasmids are circular, dsDNA elements that include one or more cloning sites for insertion of selected DNA sequences, e.g., coding sequences. Such plasmids may include a functional origin of replication and thus are replication competent, or may be replication defective.

In addition to plasmids, viral vectors (e.g., replication defective retroviruses, lentiviruses, adenoviruses and adeno-associated viruses) can also be advantageously used. A large number of such viral vectors have been developed having a broad variety of different properties. For example, such viral vectors may be replication defective retroviruses, adenoviruses and adeno-associated viruses. Techniques and procedures for producing recombinant retroviruses and for infecting cells in vitro or in vivo with such viruses are provided in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include .psi.Crip, .psi.Cre, .psi.2 and .psi.Am.

The genome of adenovirus can be manipulated such that it encodes and expresses a regulatable shRNA construct, as described herein, but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Alternatively, an adeno-associated virus vector such as that described in Tratschin et al. (1985) Mol. Cell. Biol. 5:3251-3260 can be used to express a transactivator fusion protein.

Other viral vector alternatives include lentiviral vectors. Such vectors and their preparation and use are described, for example, in U.S. Pat. Nos. 6,924,123; 6,863,884; 6,830,892; 6,818,209; 6,808,923; 6,799,657, all of which are incorporated herein in their entireties.

The vectors of the invention can advantageously include a RNAi agent-encoding (e.g., shRNA-encoding) nucleic acid operatively linked with Pol III/Pol II fusion promoters. Other elements included in the design of a particular expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The vectors described herein can be introduced into cells or tissues by any one of a variety of known methods within the art. Such methods are described for example in Sambrook et al., Molecular Cloning: A Laboratory Manual, Cold Spring Harbor Laboratory, New York (1992), which is hereby incorporated by reference. See, also, Ausubel et al., Current Protocols in Molecular Biology, John Wiley and Sons, Baltimore, Md. (1989); Hitt et al., “Construction and propagation of human adenovirus vectors,” in Cell Biology: A Laboratory Handbook, Ed. J. E. Celis., Academic Press. 2.sup.nd Edition, Volume 1, pp: 500-512, 1998; Hitt et al., “Techniques for human adenovirus vector construction and characterization,” in Methods in Molecular Genetics, Ed. K. W. Adolph, Academic Press, Orlando, Fla., Volume 7B, pp: 12-30, 1995; Hitt, et al., “Construction and propagation of human adenovirus vectors,” in Cell Biology: A Laboratory Handbook,” Ed. J. E. Celis. Academic Press. pp: 479-490, 1994, also hereby incorporated by reference. The methods include, for example, stable or transient transfection, lipofection, electroporation and infection with recombinant viral vectors. The term “transfecting” or “transfection” is intended to encompass all conventional techniques for introducing nucleic acid into host cells, including calcium phosphate co-precipitation, DEAE-dextran-mediated transfection, lipofection, electroporation and microinjection. Suitable methods for transfecting host cells can be found in Sambrook et al. (Molecular Cloning: A Laboratory Manual, 2nd Edition, Cold Spring Harbor Laboratory press (1989)), and other laboratory textbooks.

For plant cells, a Ti plasmid or viral vector is often used. For example, such plasmids and viral vectors can be used to transfect host plant cells via Agrobacterium tumefaciens-mediated transfection (for plant cells susceptible to A. tumefaciens infection), or can be directly inserted in cells, e.g., using microinjection, particle bombardment, or electroporation. In other methods, protoplasts can be made from plant cells and then transfected.

The number of host cells transformed with a nucleic acid constructs of the invention will depend, at least in part, upon the type of recombinant expression vector and the type of transfection technique used. Nucleic acid can be introduced into a host cell transiently, or for long-term expression. For long-term expression, the nucleic acid is stably integrated into the genome of the host cell or remains as a stable episomal element.

For integration of nucleic acid into host cell DNA, typically a gene is used that encodes a selectable marker (e.g., drug resistance) is introduced into the host cells along with the nucleic acid of interest. A variety of such selectable markers are commonly used, such as the drugs hygromycin and neomycin. Selectable markers can be introduced on a separate plasmid or other vector from the nucleic acid of interest or, are introduced on the same vector. Host cells transfected with a nucleic acid construct of the invention (e.g., a recombinant expression vector) and a gene for a selectable marker can be identified by selecting for cells using the selectable marker.

The present nucleic acid constructs can be introduced into eukaryotic cells growing in culture in vitro by conventional transfection techniques (e.g., calcium phosphate precipitation, DEAE-dextran transfection, electroporation, and other methods). Cells can also be transfected in vivo, for example by application of a delivery mechanism suitable for introduction of nucleic acid into cells in vivo, such as viral vectors (see e.g., Ferry, N et al. (1991) Proc. Natl. Acad. Sci. USA 88:8377-8381; and Kay, M. A. et al. (1992) Human Gene Therapy 3:641-647), adenoviral vectors (see e.g., Rosenfeld, M. A. (1992) Cell 68:143-155; and Herz, J. and Gerard, R. D. (1993) Proc. Natl. Acad. Sci. USA 90:2812-2816), receptor-mediated DNA uptake (see e.g., Wu, G. and Wu, C. H. (1988) J. Biol. Chem. 263:14621; Wilson et al. (1992) J. Biol. Chem. 267:963-967; and U.S. Pat. No. 5,166,320), direct injection of DNA (see e.g., Acsadi et al. (1991) Nature 332: 815-818; and Wolff et al. (1990) Science 247:1465-1468) or particle bombardment (see e.g., Cheng, L. et al. (1993) Proc. Natl. Acad. Sci. USA 90:4455-4459; and Zelenin, A. V. et al. (1993) FEBS Letters 315:29-32). Thus, in the present invention, cells can be transfected in vitro or ex vivo, and administered to a subject or, alternatively, cells can be directly modified in vivo.

Another aspect of the invention pertains to host cells into which a host construct of the invention has been introduced, i.e., a “recombinant host cell.” It is understood that the term “recombinant host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell, although eukaryotic cells are preferred. Exemplary eukaryotic cells include mammalian cells (such as Chinese hamster ovary cells (CHO) or COS cells). Other suitable host cells are known to those skilled in the art.

### V. Transgenic Animals

The present invention also concerns transgenic organisms, such as non-human animals, which are animals that have at least some cell that express a transgene. Such nonhuman transgenic animals can be used, for example, in screening assays designed to identify active agents or compounds, e.g., drugs, pharmaceuticals, etc., which are capable of ameliorating detrimental symptoms of selected disorders, such as disease and disorders associated with mutant or aberrant gene expression, gain-of-function mutants and neurological diseases and disorders.

A transgene is a construct that has been or is designed to be incorporated into a cell, e.g., a mammalian cell, that is incorporated in a living animal such that the construct containing the nucleotide sequence is expressed. The transgene may include a sequence (e.g., a RNAi agent-encoding sequence) that is endogenous or exogenous to the transgenic animal. A transgene may be present as an extrachromosomal element in some or all of the cells of a transgenic animal or integrated into some or all of the cells, more preferably into the germline DNA of the animal (i.e., such that the transgene is transmitted to all or some of the animal's progeny), thereby directing expression of the product of the transgene in one or more cell types or tissues of the transgenic animal. Unless clearly indicated to the contrary, reference to a transgenic animal herein will mean that the transgene is present long term as opposed to transiently, e.g., stably incorporated in the chromosomes of germline cells. In many cases, it is desirable for the transgene to be incorporated in the genome at a site such that it does not interfere with endogenous gene expression.

A present transgenic non-human animal can be, e.g., a mammal, a bird, a reptile or an amphibian. Suitable mammals for uses described herein include: rodents; ruminants; ungulates; domesticated mammals; and dairy animals. Preferred animals include: rodents, goats, sheep, camels, cows, pigs, horses, oxen, llamas, chickens, geese, and turkeys. In a preferred embodiment, the non-human animal is a mouse or a rat.

Various methods for producing transgenic animals have been described (see, e.g., Watson, J. D., et al., “The Introduction of Foreign Genes Into Mice,” in Recombinant DNA, 2d Ed., W. H. Freeman & Co., New York (1992), pp. 255-272; Gordon, J. W., Intl. Rev. Cytol. 115:171-229 (1989); Jaenisch, R., Science 240: 1468-1474 (1989); Rossant, J., Neuron 2: 323-334 (1990)). An exemplary protocol for the production of a transgenic pig can be found in White and Yannoutsos, Current Topics in Complement Research: 64th Forum in Immunology, pp. 88-94; U.S. Pat. No. 5,523,226; U.S. Pat. No. 5,573,933; PCT Application WO93/25071; and PCT Application WO95/04744. An exemplary protocol for the production of a transgenic rat can be found in Bader and Ganten, Clinical and Experimental Pharmacology and Physiology, Supp. 3:S81-S87, 1996. An exemplary protocol for the production of a transgenic cow can be found in Transgenic Animal Technology, A Handbook, 1994, ed., Carl A. Pinkert, Academic Press, Inc. An exemplary protocol for the production of a transgenic sheep can be found in Transgenic Animal Technology, A Handbook, 1994, ed., Carl A. Pinkert, Academic Press, Inc. Certain exemplary methods are set forth in more detail below.

A. Pronucleus Injection

Transgenic animals can be produced by injecting a nucleic acid construct according to the present invention into egg cells. Embryonic target cells at various developmental stages are used to introduce the transgenes. Different methods are used depending on the stage of development of the embryonal target cell(s). Exemplary methods for introducing transgenes include, but are not limited to, microinjection of fertilized ovum or zygotes (Brinster, et al., Proc. Natl. Acad. Sci. USA (1985) 82: 4438-4442), and viral integration (Jaenisch R., Proc. Natl. Acad. Sci. USA (1976) 73: 1260-1264; Jahner, et al., Proc. Natl. Acad. Sci. USA (1985) 82: 6927-6931; Van der Putten, et al., (1985) Proc. Natl. Acad. Sci. (USA) 82: 6148-6152). Procedures for embryo manipulation and microinjection are described in, for example, Manipulating the Mouse Embryo (Cold Spring Harbor Laboratory Press, Cold Spring Harbor, N.Y., 1986, the contents of which are incorporated herein by reference). Similar methods are used for production of other transgenic animals.

B. Transgenic Animals from Embryonic Stem Cells

Another method of making transgenic animals, e.g., transgenic mice, recombinant DNA molecules (e.g., constructs or transgenes) are introduced into embryonic stem (ES) cells, e.g., mouse cells. Resulting recombinant ES cells are then microinjected into mouse blastocysts using standard techniques.

In general, ES cells are obtained from pre-implantation embryos and cultured in vitro (Evans, M J., et al., Nature 292: 154156 (1981); Bradley, M. O. et al., Nature 309: 255-258 (1984); Gossler, et al., Proc. Natl. Acad. Sci. (USA) 83:9065-9069 (1986); Robertson et al., Nature 322: 445448 (1986)). Any ES cell line that is capable of integrating into and becoming part of the germ line of a developing embryo is suitable for creating germ line transmission of the construct. The ES cells can be cultured and prepared for DNA insertion using methods known in the art, e.g., as described in Robertson, Teratocarcinomas and Embryonic Stem Cells. A Practical Approach, E. J. Robertson, ed. IRL Press, Washington, D.C., 1987; in Bradley et al., Current Topics in Devel. Biol., 20:357-371, 1986; and in Hogan et al., Manipulating the Mouse Embryo: A Laboratory Manual, Cold Spring Harbor Laboratory Press, Cold Spring Harbor, N.Y., 1986, the contents of all of which are incorporated herein by reference.

Expression constructs can be introduced into the ES cells by methods known in the art, e.g., those described in Sambrook et al., Molecular Cloning. A Laboratory Manual, 2.sup.nd Ed., ed., Cold Spring Harbor laboratory Press: 1989, the contents of which are incorporated herein by reference. Exemplary methods include, but are not limited to, electroporation, microinjection, and calcium phosphate treatment methods.

Transformed ES cells are typically identified by screening for the presence of the construct. For example, ES cell genomic DNA can be examined directly. This can be accomplished, for example, by extracting the DNA from the ES cells using standard methods and probing on a Southern blot with a probe or probes designed to specifically hybridize to the transgene sequence. Genomic DNA can also be amplified by PCR with use of primers specifically designed to amplify DNA fragments of a particular size and sequence of the construct or transgene such that, only those cells containing the construct or transgene will generate DNA fragments of the proper size. In another approach, a marker gene is incorporated in the construct, and the cells tested for the presence of the marker gene. For example, for an antibiotic resistance marker gene, the cells can be cultured in the presence of an otherwise lethal concentration of antibiotic. The presence of the antibiotic selects for those cells that contain the transgene construct. If the marker gene encodes an enzyme with detectable activity (e.g., beta.-galactosidase or luciferase), the enzyme substrate can be added to the cells under suitable conditions, and the enzymatic activity can be determined as an indicator of the presence of the transgene construct.

Transgenic animals can be identified after birth by standard protocols. For example, DNA from tissue can be screened for the presence of the transgene construct, e.g., using Southern blots and/or PCR. Offspring that appear to be mosaics can be crossed to each other in order to generate homozygous animals. If it is unclear whether the offspring will have germ line transmission, they can be crossed with a parental or other strain and the offspring screened for heterozygosity. The heterozygotes are identified by Southern blots and/or PCR amplification of the DNA. The heterozygotes can then be crossed with each other to generate homozygous transgenic offspring. Homozygotes can be identified by Southern blotting of equivalent amounts of genomic DNA from offspring that are the product of this cross, as well as animals that are known heterozygotes and wild type animals. Probes to screen the Southern blots can be designed based on the sequence of the construct or transgene, or a marker gene, or both.

Other techniques for identifying and characterizing transgenic animals are known in the art. For example, western blots can be used to assess the level of expression of a gene targeted for inhibition by probing with an antibody against the targeted protein. Alternatively, an antibody against a marker gene product can be used.

The invention also concerns cells containing a present transgene derived from transgenic animals. Because certain genetic changes may occur in succeeding generations, e.g., due to mutation or recombination, such progeny cells may not be identical to the parent cell.

### VI. Construction and Testing of Pol III/Pol II Fusion Promoters

Fusion promoters can be constructed using a basal promoter from a Pol III transcribed gene along with one or more additional regulatory elements from at least one Pol II regulatory region. In addition, additional regulatory elements from the same or different Pol III gene may be incorporated in the fusion promoter. For simplicity of construction, it is advantageous to select and use regulatory elements that are 5′ to the start site.

Several Pol III promoters have been described that could be used in the present fusion promoters, such promoters include U6, H1, tRNA promoters, adenovirus VA1, and the like. For promoters that have been studied to identify basal promoter elements, such basal promoters can be used in the present fusion promoters.

For tRNA promoters, the promoter sequences necessary and sufficient for RNA polymerase III transcription are encoded in the tRNA gene and thus transcribed into the tRNA and are still deductable from sequences located in the D and T.PSI.C loop. In some cases, there are additional regulatory sequences upstream (5′) of the main body of the gene. However, most mammalian genomes encode more than 100 tRNA genes, that are redundant and many of them lack any 5′ regulatory sequences (e.g., Thomann et al. 1989 J Mol Biol 209:505-523). A database of tRNA gene compilations can be found at http://rna.wustl.edu/tRNAdb/ showing that the human genome encodes 648 tRNA genes, some of them known to be pseudogenes but a majority (496) encoding functional tRNAs (many redundantly) that are needed to encode the 20 amino acids.

Additional Pol III basal promoters can also be identified and used using conventional promoter analysis. Thus, identification of a gene as a RNA Pol III transcribed gene provides the material for identifying the corresponding basal promoter, as well as additional regulatory elements.

Similarly, a number of regulatory regions for Pol II-transcribed genes have been analyzed, and constituent regulatory elements identified. Additional Pol II regulatory regions and elements can be identified by conventional means and used in the present invention. In most cases, it is advantageous to include the element of set of elements that are demonstrated or found to be responsible for the specific regulation properties of the regulatory region.

Examples of specifically regulated Pol II-transcribed genes that can be used to provide cis-acting regulatory elements include any of the variety of such genes identified in the art. Many such genes have been described, including examples for which promoter and regulatory elements have been described.

### VII. Target Genes and Target Sites

In general, any gene can be down-regulated using RNAi in cells that contain functional RNAi machinery, and in particular such genes can be down-regulated using the present nucleic acid constructs. A large number of such inhibitions of gene expression have been described using either siRNAs or shRNAs. Targeting of such genes is also useful in connection with the present invention.

One such target gene is mutant Cu, Zn superoxide dismutase (SOD1). (See, e.g., U.S. Patent Appl. Publ. 2005013018, which is incorporated herein by reference in its entirety). Mutations in Cu, Zn superoxide dismutase (SOD1) gene cause a subset of amyotrophic lateral sclerosis, a neurodegenerative disease that leads to motor neuron degeneration, paralysis and death (Brown and Robberecht, 2001; Siddique and Lani, 2002). It has been well established that mutant SOD1 causes motor neuron degeneration by acquisition of a toxic property (Cleveland and Rothstein, 2001). However, neither the molecular basis of this toxic property nor mechanism that leads to motor neuron death is understood. Because of this incomplete understanding of the disease mechanism, rational design of therapy has not produced robust efficacious outcomes. On the other hand, because the toxicity that kills motor neurons originates from the mutated protein (Cleveland and Rothstein, 2001), decrease of the mutant protein should alleviate or even prevent the disease.

Suitable target sites for RNAi can be identified by any of a variety of methods, e.g., known to those of ordinary skill in the art. It has been found that most sites will provide at least some level of inhibition by RNAi, but some sites are found to provide substantially higher levels of inhibition. One way of identifying such “good” sites is by simple testing of potential target sites. In addition, a number of different algorithms have been designed to identify good target sites. Generally, several sites are identified using such algorithm, and then are tested for relative effectiveness.

Thus, exemplary methods for selecting suitable regions in a mRNA target are described in available publications (see, for example, Vickers et al., J. Biol. Chem. 278:7108-7118, 2003; Elbashir et al., Nature 411:494-498, 2001; Elbashir et al., Genes Dev. 15:188-200, 2001). Good target sequences are generally those sensitive to down regulation by low concentrations of siRNA. Guidelines for the design of siNA include those provided in Ambion's Technical Bulletin #506 (available from Ambion Inc., Austin, Tex.). The use of low concentrations of siRNA and avoidance of sequences that occur in alternative spliced gene products is useful for avoiding or limiting off-target, non-sequence specific inhibition. Assessing whether a gene has been downregulated, and the extent of downregulation, can be performed using, for example, real-time PCR, PCR, western blotting, flow cytometry or ELISA methods.

As an example, potential target sites in the mRNA are identified based on rational design principles, which include target accessibility and secondary structure prediction. Each of these may affect the reproducibility and degree of knockdown of expression of the mRNA target, and the concentration of siRNA required for therapeutic effect. In addition, the thermodynamic stability of the siRNA duplex (e.g., antisense siRNA binding energy, internal stability profiles, and differential stability of siRNA duplex ends) may be correlated with its ability to produce RNA interference. (Schwarz et al., Cell 115:199-208, 2003; Khvorova et al., Cell 115:209-216, 2003). Empirical rules, such as those provided by the Tuschl laboratory (Elbashir et al., Nature 411:494-498, 2001; Elbashir et al., Genes Dev. 15:188-200, 2001) are also used.

Software and internet interactive services for siRNA design are available at the Ambion and Invitrogen websites. Additional software system for design and prioritization of siRNA oligos have also been described (see, e.g., Levenkova et al., Bioinformatics 20:430-432, 2004). The Levenkova system is available on the internet and is downloadable freely for both academic and commercial purposes.

The selection of siRNA oligos can also involve uniqueness vs human sequences (i.e., a single good hit vs human Unigene, and a large difference in hybridization temperature (Tm) against the second best hit) and on GC content (i.e., sequences with % GC in the range of 40-60%).

A more detailed picture on the potential hybridization of the oligos, RNA target accessibility and secondary structure prediction can be carried out using available RNA structure prediction software, for example, Sfold software (Ding Y and Lawrence, C. E. (2004) Rational design of siRNAs with Sfold software. In: RNA Interference: from Basic Science to Drug Development. K. Appasani (Ed.), Cambridge University Press; Ding and Lawrence, Nucleic Acids Res. 29:1034-1046, 2001; Nucleic Acids Res. 31:7280-7301, 2003). Sfold is available on the internet. RNA secondary structure determination is also described in Current Protocols in Nucleic Acid Chemistry, Beaucage et al., ed, 2000, at 11.2.1-11.2.10.

In addition, certain mutations (e.g., A or U inserted or substituted at the first, second, or third positions on the 5′ end of the antisense strand of a siRNA or shRNA) insertions, have been described as providing enhanced RNAi efficiency and can be incorporated in the present constructs. Such mutations are described, for example, in U.S. Appl. Publ. 20050166272, which is incorporated herein by reference in its entirety.

### VIII. Methods of Use

The present invention is suitable for a variety of different applications, e.g., in biotechnology, gene analysis, drug identification and development, identification and development of drugs, and in medical treatment methods. For example, there is currently performed a great deal of analysis of gene function in humans as well as in other animals and other organisms. Thus, the ability to conveniently provide transgenic organisms or recombinant cells with modulation of specific genes enables the analysis of gene function, as well as the identification and evaluation of drug compounds.

In particular, by determining the effect of down-regulating specific genes in transgenic animals or cells, the biological function of those genes can be determined. Having an identified gene function allows drug targets to be validated, and for disease models to be established. Thus, specific cells may be transfected in vivo or ex vivo with recombinant vectors (e.g., viral vectors such as retrovirus vectors) encoding an RNAi agent that down-regulates the activity of a gene, for example, a gene whose activity is associated or correlated with a particular disease or condition.

In some applications it can be advantageous to determine the presence and/or level of a particular nucleic acid or polypeptides, such as the RNAi agents (e.g., siRNA or shRNA) and/or target mRNAs and/or the gene products encoded by such target mRNAs. A variety of applicable qualitative and quantitative detection methods and related techniques are known and can be used, including, for example, nucleic acid cloning and sequencing, oligonucleotide ligation, use of the polymerase chain reaction (PCR) and variations thereof, single nucleotide primer-guided extension assays, hybridization techniques using target-specific oligonucleotides, and sandwich hybridization methods.

Sequencing may be carried out with commercially available automated sequencers utilizing labeled primers or terminators, or using sequencing gel-based methods. Sequence analysis is also carried out by methods based on ligation of oligonucleotide sequences which anneal immediately adjacent to each other on a target DNA or RNA molecule (Wu and Wallace, Genomics 4: 560-569 (1989); Landren et al., Proc. Natl. Acad. Sci. 87: 8923-8927 (1990); Barany, F., Proc. Natl. Acad. Sci. 88: 189-193 (1991)). The Ligase Chain Reaction (LCR), which utilizes the thermostable Taq ligase for target amplification, is particularly useful such that the ligation reaction can be carried out at elevated reaction temperatures providing high stringency (Barany, F., PCR Methods and Applications 1: 5-16 (1991)).

Hybridization reactions may be carried out in a variety of formats, including filter-based, Southern blots, slot blots, “reverse” dot blots, solution hybridization, solid support based sandwich hybridization, bead-based, silicon chip-based and microtiter well-based hybridization formats. Specific oligonucleotide probes typically range in size between 10-1,000 bases, more commonly between 15 and 50 bases. In order to achieve a needed target discrimination using the oligonucleotide probes, hybridization reactions are generally run in the range of 20-60 degrees C., and more commonly in the range of 30-50 degrees C., with the temperature and/or salt concentrations and/or inclusion of other chaotropic agents such as formamide in the washes selected to provide optimal discrimination.

Detection of specific proteins or polypeptides is commonly performed using directly- or indirectly labeled specific antibodies, e.g., monoclonal or polyclonal antibodies, or fragments thereof. Examples of such labels include fluorescent moieties, colorometric moieties, light scattering moieties, and radioisotopes. Those of ordinary skill in the art are familiar with carrying out such detection.

The general detection methods mentioned above, as well as other methods, can be used in testing and/or using the present nucleic acid constructs.

A. Screening, Assays, and Therapeutic Agent Testing

The present invention is applicable to use in screening assays, e.g., to identify and/or analyze potential pharmacological agents, e.g. identifying new pharmacological agents from a library of test compounds and/or characterizing mechanisms of action and/or side effects of compounds that have known pharmacological activities.

Thus, the present invention concerns materials and methods for carrying out a variety of biological assays and/or drug screening assays using cells or organisms that express agents, especially RNAi agents, from the present Pol III/Pol II fusion promoters. Generally such cells are eukaryotic cells (e.g., animal or plant cells) and/or such organisms are eukaryotic organisms (e.g., non-human transgenic animals).

Such assays and tests generally involve expressing a nucleic acid sequence (e.g., an RNAi agent-encoding sequence) in a cell or organism and determining at least one effect of that expression. The assay may be conducted to test or assay a single or small number of RNAi agents, or can be carried out in large scale assaying, e.g., for compounds in a compound library, such as assaying or testing at least 10, 100, 1000, 10⁴, 10⁵, 10⁶ compounds.

Assays or tests involving determination of the effect(s) of RNAi agent expression can advantageously also involve determining or comparing the effect(s) of the absence of the RNAi agent expression, the presence of a positive and/or negative control compound, and/or the presence of one or more test compounds. Typically, such assays or test involve determining pharmacological properties of the RNAi agent and/or other test compounds.

Test compounds can be obtained in many different ways, e.g., using any of the numerous approaches in compound library methods known in the art. For example, libraries can be commercially available compound libraries, libraries constructed from commercially available compounds, custom compound libraries, synthetic compound libraries, and natural product libraries, e.g., produced by bacteria, yeast, and/or fungi.

One broad category of libraries and library methods are combinatorial library methods including without limitation: biological libraries; spatially addressable parallel solid phase or solution phase libraries; synthetic library methods requiring deconvolution; the ‘one-bead one-compound’ library method; and synthetic library methods using affinity chromatography selection. (Lam, K. S. (1997) Anticancer Drug Des. 12:145). Such libraries can be peptide and/or peptide analog, oligonucleotide and/or oligonucleotide analog, and/or small molecule libraries.

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented, for example, in solution (e.g., Houghten (1992) Biotechniques 13:412-421), on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.)).

Additional compounds or agents identified according to screening assays can be further tested and/or developed and/or used therapeutically or prophylactically either alone or in combination, for example, with an RNAi agent of the invention.

B. Functional Genomics and/or Proteomics

Certain applications for the cells and organism of the invention include the analysis of gene expression profiles and/or proteomes. In may cases, such analysis involves knock-out or knock-down of a target gene, and determining a phenotypic change as in indicator of gene function or effect of inhibition. Alternatively, such analysis can be directed to a variant or mutant form of one or several target proteins, where the variant or mutant forms are reintroduced into the cell or organism by an exogenous target nucleic acid as described above. The combination of knockout of an endogeneous gene and rescue by using mutated, e.g., partially deleted exogenous target has certain advantages such as assisting in identifying functional domains of the targeted protein. Such analysis can be carried out for multiple cell types and/or tissues and/or organisms. These cells and/or organisms are generally selected from: (i) a control cell or control organism without target gene inhibition, (ii) a cell or organism with target gene inhibition and (iii) a cell or organism with target gene inhibition plus target gene complementation by an exogenous target nucleic acid.

Such RNA knockout complementation method may be used for its preparative purposes, e.g., for the affinity purification of proteins or protein complexes from eukaryotic cells, particularly mammalian cells and more particularly human cells. In this embodiment of the invention, the exogenous target nucleic acid preferably codes for a target protein which is fused to an affinity tag. This method is suitable for functional proteome analysis in mammalian cells, particularly human cells. Another utility of the present invention is a method of identifying gene function in an organism by using an RNA molecule to inhibit the activity of a target gene of previously unknown function. Instead of the time consuming and laborious isolation of mutants by traditional genetic screening, functional genomics would envision determining the function of uncharacterized genes by employing the invention to reduce the amount and/or alter the timing of target gene activity. The invention can be used in determining potential targets for pharmaceutics, understanding normal and pathological events associated with development, determining signaling pathways responsible for postnatal development/aging, and the like.

Creation of cells/organisms containing the target gene allows the present invention to be used in high throughput screening (HTS). For example, solutions containing such cells containing RNAi agent capable of inhibiting the different expressed genes can be placed into individual wells positioned on a microtiter plate as an ordered array, and intact cells/organisms in each well can be assayed for any changes or modifications in phenotype, behavior, and/or development corresponding to inhibition of target gene activity. Such screening is amenable to cells as well as to small subjects that can be processed in large number, for example: arabidopsis, drosophila, fingi, nematodes, viruses, zebrafish, plants, and tissue culture cells derived from various organisms, such as mammals. A nematode or other organism that produces a calorimetric, fluorogenic, or luminescent signal in response to a regulated promoter (e.g., transfected with a reporter gene construct) can be assayed in an HTS format.

HTS can be used to identify and/or characterize new drug targets. The potential drug targets may also be validated using the present invention. For example, a particular disease phenotype may be induced by a gene mutation or a chemical. RNAi may be used to down-regulate genes and some of these down-regulations can lead to the reversal of the disease phenotype or other phenotypic change indicting a therapeutic or prophylactic effect. These genes are potential drug targets.

C. Treatment Methods

The present invention provides RNAi agent-expressing constructs that are therapeutically useful (e.g., in certain prophylactic and/or therapeutic applications). For example, such agents can be used as prophylactic and/or therapeutic agents in the treatment of diseases or disorders associated with unwanted or aberrant expression of the corresponding target gene.

Thus, the invention provides prophylactic methods of treating a subject at risk of (or susceptible to) a disease or disorder, for example, a disease or disorder associated with aberrant or unwanted target gene expression or activity or susceptible to an infection. Administration of a prophylactic agent can occur prior to the manifestation of symptoms characteristic of the disease or condition, such that a disease or disorder is prevented or delayed in its progression or reduced in severity.

Likewise, the invention provides therapeutic methods of treating a subject having a disease or disorder, for example, a disease or disorder associated with aberrant or unwanted target gene expression or activity or expression from an infective agent.

Knowledge of RNAi agents and their targets thus allows specific inhibition of such target genes to treat any of a number of disorders (including cancer, inflammation, neuronal disorders, etc.) using the present constructs and methods.

For such prophylactic and therapeutic methods of treatment, the treatments may be tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, the invention also provides methods for tailoring an individual's prophylactic or therapeutic treatment with the present constructs and methods according to that individual's drug response genotype. Pharmacogenomics allows a clinician or physician to target prophylactic or therapeutic treatments to patients who will most benefit from the treatment and to avoid treatment of patients who will experience toxic drug-related side effects.

### IX. Pharmaceutical Compositions

For preparation of pharmaceutical compositions containing the present RNAi agents for prophylactic and/or therapeutic treatments, the agents are routinely incorporated into pharmaceutical compositions suitable for administration. Such compositions include the nucleic acid molecule and commonly include a pharmaceutically acceptable carrier, and may include additional components. The use of such carriers for pharmaceutically active substances is well known in the art. Any conventional media or agent incompatible with the active compound can be used in the present pharmaceutical compositions. The additional components can, for example, include additional or supplementary active compounds.

A present pharmaceutical composition is formulated to be compatible with its intended route of administration, for example, parenteral, e.g., intravenous, intradermal, subcutaneous, intraperitoneal, intramuscular, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The composition can be aliquoted or packaged in ampules, disposable syringes, single or multiple dose vials made of glass or plastic, bottles, and the like. Preferably the composition is sterile at a medically acceptable level in view of the intended route of administration. In some cases, the pharmaceutical composition is approved by a governmental drug regulatory agency (e.g., the U.S. FDA) for administration to a particular class of subject, such as human subjects.

Pharmaceutical compositions adapted for injection include, for example, sterile aqueous solutions (where water soluble) or dispersions and sterile powders for the extemporaneous preparation of sterile injectable solutions or dispersion. For intravenous administration, suitable carriers include, for example, physiological saline, bacteriostatic water, Cremophor EL™. (BASF, Parsippany, N.J.) and phosphate buffered saline (PBS). In all cases, the composition should be sterile and should be fluid or convertible to a fluid at least sufficient for easy syringability. The composition and/or nucleic acid constructs should be stable under the conditions of manufacture and storage and should be preserved against the contaminating action of microorganisms such as bacteria and fungi.

The carrier can be a solvent or dispersion medium containing, for example, water, ethanol, polyol (for example, glycerol, propylene glycol, and liquid polyethylene glycol, and the like), and suitable mixtures thereof. Fluidity can be maintained, for example, by the use of a coating such as lecithin, by the maintenance of the required particle size in the case of dispersions and by the use of surfactants.

Preservatives against microorganisms can include various antibacterial and antifungal agents, for example, parabens, chlorobutanol, phenol, ascorbic acid, thimerosal, and the like.

In many cases, it will be desirable for the composition to be isotonic to blood. This can be accomplished using various isotonic agents, for example, sugars, polyalcohols such as manitol, sorbitol, sodium chloride in the composition.

Delayed or extended absorption of the injectable compositions can be desirable and can be achieved by including in the composition an agent which delays absorption, for example, aluminum monostearate and gelatin, or by coating micro- or nano-particles of active agent in the composition with materials that delayed or extended release of components.

Sterile injectable solutions can be prepared, for example, by solubilizing or suspending the active compound in the required amount in an appropriate solvent with one or a combination of additional ingredients. Typically creation of such solution or suspension is followed by sterile filtration. Generally, dispersions are prepared by incorporating the active compound into a sterile vehicle which contains a basic dispersion medium and the other desired ingredients. In the case of sterile powders for the preparation of sterile injectable solutions, the preparation is dried, e.g., by vacuum drying and/or freeze-drying.

Compositions for oral administration typically include an inert or edible diluent or edible carrier. Such compositions can be formulated in various ways, e.g., in liquid, capsule, or tablet form. Pharmaceutically compatible binding agents, and/or adjuvant materials can be included as part of the composition. The tablets, pills, capsules, troches and the like can contain any one or more of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For inhalation administration, the compounds are delivered in the form of a wet or dry aerosol spray, e.g., from a pressured container or dispenser which contains a suitable propellant, e.g., a gas such as carbon dioxide, or a nebulizer.

Systemic administration can also be by transmucosal or transdermal routes. For transmucosal or transdermal administration, penetrants appropriate to the barrier to be permeated are typically used in the formulation. A number of such penetrants are generally known in the art, and include, for example, for transmucosal administration, detergents, bile salts, and fusidic acid derivatives.

Transmucosal administration can be accomplished through the use of nasal sprays or suppositories (e.g., using conventional suppository bases such as cocoa butter and other glycerides). For transdermal administration, the active compounds are formulated into ointments, salves, gels, or creams as generally known in the art.

Such compositions can also be formulated with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. The materials can also be obtained commercially, e.g., from Alza Corporation and Nova Pharmaceuticals, Inc. Liposomal suspensions (including liposomes targeted to particular cells (e.g., targeted to infected cells) with monoclonal antibodies) can also be used to prepare pharmaceutical compositions. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of active compounds and pharmaceutical compositions can be determined by standard pharmaceutical procedures in cell cultures or experimental animals. For example, such procedures are routinely applied for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are generally preferred.

The data obtained from the cell culture assays and animal studies can be used in formulating a range of dosage for use in humans or other intended subject. The dosage of such compounds is usually selected to produce a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. Thus, for example, a dose may be initially established in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography, or by other suitable analysis method adapted for the compound of interest.

### X. Exemplary Delivery Methods and Compositions

A number of delivery methods applicable to nucleic acid molecules, and particularly to transcribable nucleic acid molecules, have been described, and additional ones are being developed. All such methods and the associated compositions are applicable to the present invention. Such delivery typically utilizes naked linear nucleic acid, viral vectors, or plasmid vectors.

### EXAMPLES

The ability of Pol III/Pol II fusion promoters to specifically regulate expression of functionally linked coding sequences, particularly RNAi agents, was demonstrated using cell-type specific expression based on the cell-type specific regulatory elements from the Pol II regulatory region of GFAP. The sequences of various constructs are provided as SEQ ID NOs:1-7. The following examples provide an illustrative description of the creation and expression of exemplary nucleic acid constructs, but are not intended to and do not limit the invention.

### Example 1

### Construction of Pol III/Pol II Fusion Promoters, Associated Constructs, and Vectors

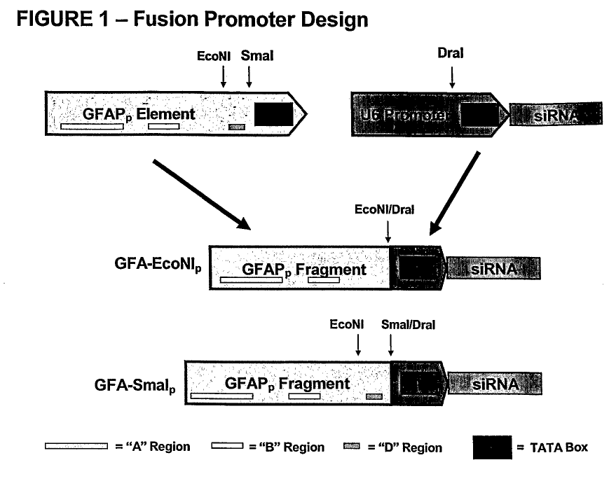

To test whether tissue specific RNAi promoters could be created using a chimeric promoter, promoters that are the fusion of GFAP and U6 promoters were created. Promoter studies have revealed that the GFAP promoter is composed of several elements that are involved in tissue specific expression including an A, B and a D region. These cis-acting elements are instrumental in the transcription program of the human GFAP promoter. Two promoters were created: one that included the A and B elements of GFAP linked to the core promoter of U6 termed GFAP-EcoNIp and one that included the A, B and D elements of the GFAP promoter linked to the core promoter of U6 termed GFAP-SmaIp (see FIG. 1).

In each of the two promoters, the GFAP elements are placed upstream of the core promoter of U6. The core promoter of U6 has been shown to include the TATA box region and the PSE. Thus, both chimeric promoters contain elements of the RNA polymerase type II promoter and elements (specifically including the core promoter region) of an RNA polymerase type III promoter. These are named according to the restriction site used in their creation, i.e. pGliaSmaI and pGliaEcoNI. For initial testing purposes, RNAi directed against eGFP was included. Two additional plasmids were created that included the RNAi against eGFP termed pGliaSmaI-eGFP and pGliaEcoNI-eGFP (see FIG. 1). Finally, a dicistronic plasmid was created with expression cassettes for both eGFP and HcRed1. The design of the study was to cotransfect the dicistronic plasmid (i.e. peGFP/HcRed1) with the chimeric promoter containing plasmids and assay the expression of eGFP for knockdown and HcRed1 as a control.

Several additional vectors were created (maps not shown). Four more plasmids were created where the chimeric promoters (plus/minus RNAi against eGFP) was cloned into peGFP/HcRed1. These vectors were intended to allow testing of the idea without the use of cotransfection. Alternately, four additional vectors were created where the LacZ expression cassette was cloned into the plasmids containing the chimeric vectors. The purpose of these vectors was to allow for monitoring of the transfection efficiency of the chimeric promoter containing vectors in co-transfection experiments. Using this system, it is possible to monitor the transfection of both plasmids, i.e. HcRed1 expression to monitor the delivery of eGFP and LacZ to monitor the delivery of the chimeric promoter.

The vectors were constructed generally as follows. A dicistronic vector with both eGFP and HcRed1 expression cassettes (termed peGFP-HcRed1) was created in several steps. First, pHcRed1-RNAi was created by ligating the HcRed1-containing fragment of pHcRed1-N1 (Clontech) doubly digested with AgeI (blunted) and NotI into the backbone fragment of pHygeEGFP (Clontech) doubly digested with NheI (blunt) and NotI. Second, peGFP-RNAi was created by ligating the eGFP containing fragment of peGFP-1 (Clontech) doubly digested with SmaI and NotI into the backbone fragment of pHygeGFP (Clontech) doubly digested with NheI (blunted) and NotI. peGFP-HcRed1 was created by ligating the CMVie-eGFP-polyA cassette of peGFP-RNAi doubly digested with BglII and BamHI into pHcRed1-RNAi digested with BamHI and calf alkaline phosphatase.

Two RNAi chimeric promoters targeting glial cells were created using the U6 promoter and the glial fibrillary acidic protein (GFAP) promoter (pGfa2; Brenner et al). A ˜150 bp fragment of pU6-eGFP shRNA (Shi et al) doubly digested with DraI and BamHI was ligated into the backbone fragment of pGfa2 doubly digested with SmaI and BamHI to create pGFAP-SmaI-eGFP. Similarly, a control vector (pGFAP-SmaI-Control) was created by ligating an ˜150 bp fragment of pU6-control (Shi et al) into the backbone fragment of pGfa2 doubly digested with SmaI and BamHI. To make both pGFAP-EcoNI-eGFP and pGFAP-EcoNI-Control, the ˜150 bp fragment of pU6-eGFP and pU6-control were ligated (respectively) into the backbone fragment of pGfa2 doubly digested with EcoNI (blunted) and BamHI.

Four additional plasmids were created by ligating the BglII/BamHI fragment of pGfaSmaI-control, pGfaSmaI-eGFP, pGfaEcoNI-control, or pGfaEcoNI-eGFP into peGFP/HcRed1 doubly digested with BamHI and alkaline phosphatase. Directionality was determined by restriction analysis and clones where the promoter direction of the chimeric promoter and the eGFP expression cassette were opposed were selected for testing.

A final set of four vectors were created by first cloning the LacZ containing BamHI fragment of pGfa2 (Brenner et al) into the BamHI/BclI backbone fragment of pIRES-eYFP (Clontech) to create pCMV-LacZ. Finally, the chimeric promoter containing fragments of pGfaSmaI-control, pGfaSmaI-eGFP, pGfaEcoNI-control, or pGfaEcoNI-eGFP doubly digested with BglII/BamHI were individually ligated into pCMV-LacZ doubly digested with BglII and alkaline phosphatase. These vectors, named pGfaSmaI-control-LacZ, pGfaSmaI-eGFP-LacZ, pGfaEcoNI-control-LacZ, or pGfaEcoNI-eGFP-LacZ, are useful in monitoring transfection efficiency of the chimeric promoters.

### Example 2

### Transfection of Cells with Vectors Containing Pol III/Pol II Fusion Promoters Linked with shRNA Coding Sequence

C6 glioma cells, Hela S3 cells, and HepG2 cells were cultured according to standard protocols using DMEM supplemented with 10% FBS. The day before transfection, cells were plated onto 6-well plates to achieve 60-70% density the following day for transfection.

Cells were transfected with Lipofectamine 2000 (Invitrogen) transfection reagent according to manufacturer's protocols. Briefly, 300 ng of peGFP-HcRed1 plus 3-6 micrograms of the shRNA expression (or control) plasmids were diluted in 100 microliters of OptiMem (Invitrogen). Separately, 7 microliters of Lipofectamine 2000 was diluted into 100 microliters of OptiMem. These two dilutions were combined and allowed to incubate for 20 minutes. During this time, the DMEM culture media was rinsed out of each well and replaced by 1 milliliter of OptiMem. After the twenty minute incubation for liplex formation, the lipoplexes were added to each well. After eight hours of incubation, another milliliter of OptiMem was added to each well to bring the total to 2.2 milliliters. After 24 hours, the culture media was exchanged for DMEM with 10% FBS and further experiments were conducted (i.e. either fluorescent microscopy or Western Blot analysis) at 24-48 hours after transfection.

### Example 3

### Expression and Analysis of shRNA

Preliminary analysis demonstrated that the exemplary constructs provided cell-specific expression of the linked coding sequences encoding shRNAs. Cells were grown generally as described above.

Fluorescent Microscopy

To test the plasmids, C6 glioma cells and Hela S3 cells plated in 6-well plates were transfected with different combinations of the created plasmids. The media of cells grown in the six-well plates was exchanged with phosphate buffered saline containing calcium and magnesium and the plates were loaded onto an inverted microscope (Olympus IX70). Images were captured with ImagePro imaging software and hardware (Media Cybernetics), followed by image manipulation with Adobe Photoshop 6.0 (Adobe Systems, San Jose, Calif.). Representative images from several cell fields were captured.

Cotransfection of both peGFP/HcRed1 and the chimeric promoters (control and RNAi to eGFP) revealed that the chimeric promoters containing the D element of the GFAP promoter efficiently silenced the expression of eGFP in C6 glial cells. Further, there was no detectable silencing in Hela S3 cells. In contrast, there was no detectable silencing by the chimeric promoter that did not contain the D element in either cell line. In both cases, the visualization of HcRed1 was suboptimal so that the equal delivery of the peGFP/HcRed1 to both the control and RNAi wells could not be demonstrated fully. On the other hand, the experiment worked multiple times and peGFP/HcRed1 was diluted into a mastermix that was aliquoted into separate tubes before the addition of the chimeric promoter plasmids to ensure equal delivery.

The plasmids that contained the chimeric promoter and both eGFP and HcRed1 expression cassettes were also tested (data not shown). In these experiments, C6 glioma cells and Hela S3 cells were transfected with the single plasmid and eGFP and HcRed1 expression was visualized. However, no detectable difference eGFP fluorescence could be noted between any of the wells. Given the requirement of a copy excess of 20:1 of RNAi promoter containing plasmids to gene containing plasmid reported, it is not surprising that this did not reveal any difference. Future experiments to exchange the promoter of the eGFP expression cassette from the very active CMVie promoter to a less active promoter were considered and will be performed in the near future.

Western Blot Analysis

In order to confirm and further test the expression results determined by fluorescent microscopy, protein levels were qualitatively tested using Westerns. These experiments were carried out in the same manner as for the fluorescent microscopy analysis by cotransfecting the chimeric promoters with peGFP/HcRed1. In most cases, fluorescent microscopy was also used to characterize the experiment, but then the Western analysis was performed on each well to obtain a more sensitive measure of the effectiveness of each chimeric promoter at silencing eGFP.

Cells were cultured generally as described above. The media was removed from each well of the 6-well plate and lysis buffer was added to each well and the cells were lysed on ice. Cells were scraped from each well and Western blotting was used to examine the expression of eGFP.

As with the fluorescent microscopy, it was found that the D element was required for efficient promoter activity in C6 glioma cells. The higher sensitivity allowed for the detection of some activity in the Hela S3 cells which was to be expected. Cotransfection of the peGFP/HcRed1 promoter with pGliaSmaI-eGFP displayed silencing of eGFP protein expression compared to HcRed1 in C6 glioma cells. In contrast, pGliaEcoNI-eGFP was not nearly as efficient at silencing eGFP protein expression.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to the regulatory elements included in the constructs and in methods for delivering such constructs to cells and organisms. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

Thus, additional embodiments are within the scope of the invention and within the following claims.

TABLE 1
pSilencer 1.0 (length 3292 bp)

VERSION pDRAW 1.0 beta
DNAname pSilencer 1.0
IScircular YES
Sequence . . .

| | |
|---|---|
| 1 | CTAAATTGTA AGCGTTAATA TTTTGTTAAA ATTCGCGTTA AATTTTTGTT |
| 51 | AAATCAGCTC ATTTTTTAAC CAATAGGCCG AAATCGGCAA AATCCCTTAT |
| 101 | AAATCAAAAG AATAGACCGA GATAGGGTTG AGTGTTGTTC CAGTTTGGAA |
| 151 | CAAGAGTCCA CTATTAAAGA ACGTGGACTC CAACGTCAAA GGGCGAAAAA |
| 201 | CCGTCTATCA GGGCGATGGC CCACTACGTG AACCATCACC CTAATCAAGT |
| 251 | TTTTTGGGGT CGAGGTGCCG TAAAGCACTA AATCGGAACC CTAAAGGGAG |
| 301 | CCCCCGATTT AGAGCTTGAC GGGGAAAGCC GGCGAACGTG GCGAGAAAGG |
| 351 | AAGGGAAGAA AGCGAAAGGA GCGGGCGCTA GGGCGCTGGC AAGTGTAGCG |
| 401 | GTCACGCTGC GCGTAACCAC CACACCCGCC GCGCTTAATG CGCCGCTACA |
| 451 | GGGCGCGTCC CATTCGCCAT TCAGGCTGCG CAACTGTTGG GAAGGGCGAT |
| 501 | CGGTGCGGGC CTCTTCGCTA TTACGCCAGC TGGCGAAAGG GGGATGTGCT |
| 551 | GCAAGGCGAT TAAGTTGGGT AACGCCAGGG TTTTCCCAGT CACGACGTTG |
| 601 | TAAAACGACG GCCAGTGAGC GCGCGTAATA CGACTCACTA TAGGGCGAAT |
| 651 | TGGGTACCCG CTCTAGAACT AGTGGATCCG ACGCCGCCAT CTCTAGGCCC |
| 701 | GCGCCGGCCC CCTCGCACAG ACTTGTGGGA GAAGCTCGGC TACTCCCCTG |
| 751 | CCCCGGTTAA TTTGCATATA ATATTTCCTA GTAACTATAG AGGCTTAATG |
| 801 | TGCGATAAAA GACAGATAAT CTGTTCTTTT TAATACTAGC TACATTTTAC |
| 851 | ATGATAGGCT TGGATTTCTA TAAGAGATAC AAATACTAAA TTATTATTTT |
| 901 | AAAAAACAGC ACAAAAGGAA ACTCACCCTA ACTGTAAAGT AATTGTGTGT |
| 951 | TTTGAGACTA TAAATATCCC TTGGAGAAAA GCCTTGTTTG GGCCCCCCCT |
| 1001 | CGAGGTCGAC GGTATCGATA AGCTTGATAT CGAATTCCTG CAGCCCGGGG |
| 1051 | GATCCACTAG TTCTAGAGCG GCCGCCACCG CGGTGGAGCT CCAGCTTTTG |
| 1101 | TTCCCTTTAG TGAGGGTTAA TTGCGCGCTT GGCGTAATCA TGGTCATAGC |
| 1151 | TGTTTCCTGT GTGAAATTGT TATCCGCTCA CAATTCCACA CAACATACGA |
| 1201 | GCCGGAAGCA TAAAGTGTAA AGCCTGGGGT GCCTAATGAG TGAGCTAACT |
| 1251 | CACATTAATT GCGTTGCGCT CACTGCCCGC TTTCCAGTCG GGAAACCTGT |
| 1301 | CGTGCCAGCT GCATTAATGA ATCGGCCAAC GCGCGGGGAG AGGCGGTTTG |
| 1351 | CGTATTGGGC GCTCTTCCGC TTCCTCGCTC ACTGACTCGC TGCGCTCGGT |
| 1401 | CGTTCGGCTG CGGCGAGCGG TATCAGCTCA CTCAAAGGCG GTAATACGGT |
| 1451 | TATCCACAGA ATCAGGGGAT AACGCAGGAA AGAACATGTG AGCAAAAGGC |
| 1501 | CAGCAAAAGG CCAGGAACCG TAAAAAGGCC GCGTTGCTGG CGTTTTTCCA |
| 1551 | TAGGCTCCGC CCCCCTGACG AGCATCACAA AAATCGACGC TCAAGTCAGA |
| 1601 | GGTGGCGAAA CCCGACAGGA CTATAAAGAT ACCAGGCGTT TCCCCCTGGA |
| 1651 | AGCTCCCTCG TGCGCTCTCC TGTTCCGACC CTGCCGCTTA CCGGATACCT |
| 1701 | GTCCGCCTTT CTCCCTTCGG GAAGCGTGGC GCTTTCTCAT AGCTCACGCT |
| 1751 | GTAGGTATCT CAGTTCGGTG TAGGTCGTTC GCTCCAAGCT GGGCTGTGTG |
| 1801 | CACGAACCCC CCGTTCAGCC CGACCGCTGC GCCTTATCCG GTAACTATCG |
| 1851 | TCTTGAGTCC AACCCGGTAA GACACGACTT ATCGCCACTG GCAGCAGCCA |
| 1901 | CTGGTAACAG GATTAGCAGA GCGAGGTATG TAGGCGGTGC TACAGAGTTC |
| 1951 | TTGAAGTGGT GGCCTAACTA CGGCTACACT AGAAGAACAG TATTTGGTAT |
| 2001 | CTGCGCTCTG CTGAAGCCAG TTACCTTCGG AAAAAGAGTT GGTAGCTCTT |
| 2051 | GATCCGGCAA ACAAACCACC GCTGGTAGCG GTGGTTTTTT TGTTTGCAAG |
| 2101 | CAGCAGATTA CGCGCAGAAA AAAAGGATCT CAAGAAGATC CTTTGATCTT |
| 2151 | TTCTACGGGG TCTGACGCTC AGTGGAACGA AAACTCACGT TAAGGGATTT |
| 2201 | TGGTCATGAG ATTATCAAAA AGGANNTTCA CCTAGATCCT TTTAAATTAA |
| 2251 | AAATGAAGTT TTAAATCAAT CTAAAGTATA TATGAGTAAA CTTGGTCTGA |
| 2301 | CAGTTACCAA TGCTTAATCA GTGAGGCACC TATCTCAGCG ATCTGTCTAT |
| 2351 | TTCGTTCATC CATAGTTGCC TGACTCCCCG TCGTGTAGAT AACTACGATA |
| 2401 | CGGGAGGGCT TACCATCTGG CCCCAGTGCT GCAATGATAC CGCGAGACCC |
| 2451 | ACGCTCACCG GCTCCAGATT TATCAGCAAT AAACCAGCCA GCCGGAAGGG |
| 2501 | CCGAGCGCAG AAGTGGTCCT GCAACTTTAT CCGCCTCCAT CCAGTCTATT |
| 2551 | AATTGTTGCC GGGAAGCTAG AGTAAGTAGT TCGCCAGTTA ATAGTTTGCG |
| 2601 | CAACGTTGTT GCCATTGCTA CAGGCATCGT GGTGTCACGC TCGTCGTTTG |
| 2651 | GTATGGCTTC ATTCAGCTCC GGTTCCCAAC GATCAAGGCG AGTTACATGA |
| 2701 | TCCCCCATGT TGTGCAAAAA AGCGGTTAGC TCCTTCGGTC CTCCGATCGT |
| 2751 | TGTCAGAAGT AAGTTGGCCG CAGTGTTATC ACTCATGGTT ATGGCAGCAC |
| 2801 | TGCATAATTC TCTTACTGTC ATGCCATCCG TAAGATGCTT TTCTGTGACT |
| 2851 | GGTGAGTACT CAACCAAGTC ATTCTGAGAA TAGTGTATGC GGCGACCGAG |
| 2901 | TTGCTCTTGC CCGGCGTCAA TACGGGATAA TACCGCGCCA CATAGCAGAA |
| 2951 | CTTTAAAAGT GCTCATCATT GGAAAACGTT CTTCGGGGCG AAAACTCTCA |
| 3001 | AGGATCTTAC CGCTGTTGAG ATCCAGTTCG ATGTAACCCA CTCGTGCACC |
| 3051 | CAACTGATCT TCAGCATCTT TTACTTTCAC CAGCGTTTCT GGGTGAGCAA |
| 3101 | AAACAGGAAG GCAAAATGCC GCAAAAAAGG GAATAAGGGC GACACGGAAA |
| 3151 | TGTTGAATAC TCATACTCTT CCTTTTTCAA TATTATTGAA GCATTTATCA |
| 3201 | GGGTTATTGT CTCATGAGCG GATACATATT TGAATGTATT TAGAAAAATA |
| 3251 | AACAAATAGG GGTTCCGCGC ACATTTCCCC GAAAAGTGCC AC |

TABLE 2
GFAP EcoNI Promoter (Length 2129)

VERSION pDRAW 1.0 beta
DNAname New DNA entry
IScircular NO
Sequence . . .

| | |
|---|---|
| 1 | AGATCTGAGC TCCCACCTCC CTCTCTGTGC TGGGACTCAC AGAGGGAGAC |
| 51 | CTCAGGAGGC AGTCTGTCCA TCACATGTCC AAATGCAGAG CATACCCTGG |
| 101 | GCTGGGCGCA GTGGCGCACA ACTGTAATTC CAGCACTTTG GGAGGCTGAT |
| 151 | GTGGAAGGAT CACTTGAGCC CAGAAGTTCT AGACCAGCCT GGGCAACATG |
| 201 | GCAAGACCCT ATCTCTACAA AAAAAGTTAA AAAATCAGCC ACGTGTGGTG |
| 251 | ACACACACCT GTAGTCCCAG CTATTCAGGA GGCTGAGGTG AGGGGATCAC |
| 301 | TTAAGGCTGG GAGGTTGAGG CTGCAGTGAG TCGTGGTTGC GCCACTGCAC |
| 351 | TCCAGCCTGG GCAACAGTGA GACCCTGTCT CAAAAGACAA AAAAAAAAAA |
| 401 | AAAAAAAAAA AGAACATATC CTGGTGTGGA GTAGGGGACG CTGCTCTGAC |
| 451 | AGAGGCTCGG GGGCCTGAGC TGGCTCTGTG AGCTGGGGAG GAGGCAGACA |
| 501 | GCCAGGCCTT GTCTGCAAGC AGACCTGGCA GCATTGGGCT GGCCGCCCCC |
| 551 | CAGGGCCTCC TCTTCATGCC CAGTGAATGA CTCACCTTGG CACAGACACA |
| 601 | ATGTTCGGGG TGGGCACAGT GCCTGCTTCC CGCCGCACCC CAGCCCCCCT |
| 651 | CAAATGCCTT CCGAGAAGCC CATTGAGCAG GGGGCTTGCA TTGCACCCCA |
| 701 | GCCTGACAGC CTGGCATCTT GGGATAAAAG CAGCACAGCC CCCTAGGGGC |
| 751 | TGCCCTTGCT GTGTGGCGCC ACCGGCGGTG GAGAACAAGG CTCTATTCAG |
| 801 | CCTGTGCCCA GGAAAGGGGA TCAGGGGATG CCCAGGCATG GACAGTGGGT |
| 851 | GGCAGGGGGG GAGAGGAGGG CTGTCTGCTT CCCAGAAGTC CAAGGACACA |
| 901 | AATGGGTGAG GGGACTGGGC AGGGTTCTGA CCCTGTGGGA CCAGAGTGGA |
| 951 | GGGCGTAGAT GGACCTGAAG TCTCCAGGGA CAACAGGGCC CAGGTCTCAG |
| 1001 | GCTCCTAGTT GGGCCCAGTG GCTCCAGCGT TTCCAAACCC ATCCATCCCC |
| 1051 | AGAGGTTCTT CCCATCTCTC CAGGCTGATG TGTGGGAACT CGAGGAAATA |
| 1101 | AATCTCCAGT GGGAGACGGA GGGGTGGCCA GGGAAACGGG GCGCTGCAGG |
| 1151 | AATAAAGACG AGCCAGCACA GCCAGCTCAT GTGTAACGGC TTTGTGGAGC |
| 1201 | TGTCAAGGCC TGGTCTCTGG GAGAGAGGCA CAGGGAGGCC AGACAAGGAA |
| 1251 | GGGGTGACCT GGAGGGACAG ATCCAGGGGC TAAAGTCCTG ATAAGGCAAG |
| 1301 | AGAGTGCCGG CCCCCTCTTG CCCTATCAGG ACCTCCACTG CCACATAGAG |
| 1351 | GCCATGATTG ACCCTTAGAC AAAGGGCTGG TGTCCAATCC CAGCCCCCAG |
| 1401 | CCCCAGAACT CCAGGGAATG AATGGGCAGA GAGCAGGAAT GTGGGACATC |
| 1451 | TGTGTTCAAG GGAAGGACTC CAGGAGTCTG CTGGGAATGA GGCCTAGTAG |
| 1501 | GAAATGAGGT GGCCCTTGAG GGTACAGAAC AGGTTCATTC TTCGCCAAAT |
| 1551 | TCCCAGCACC TTGCAGGCAC TTACAGCTGA GTGAGATAAT GCCTGGGTTA |
| 1601 | TGAAATCAAA AAGTTGGAAA GCAGGTCAGA GGTCATCTGG TACAGCCCTT |
| 1651 | CCTTCCCTTT TTTTTTTTTT TTTTTTGTGA GACAAGGTCT CTCTCTGTTG |
| 1701 | CCCAGGCTGG AGTGGCGCAA ACACAGCTCA CTGCAGCCTC AACCTACTGG |
| 1751 | GCTCAAGCAA TCCTCCAGCC TCAGCCTCCC AAAGTGCTGG GATTACAAGC |
| 1801 | ATGAGCCACC CCACTCAGCC CTTTCCTTCC TTTTTAATTG ATGCATAATA |
| 1851 | ATTGTAAGTA TTCATCATGG TCCAACCAAC CCTTTCTTGA CCCACCTTCC |
| 1901 | TAGAGAGAGG GTCCTCTTGC TTCAGCGGTC AGGGCCCCAG ACCCATGGTC |
| 1951 | TGGCTCCAGG TACCACCTGC CTCTAAAAAA CAGCACAAAA GGAAACTCAC |
| 2001 | CCTAACTGTA AAGTAATTGT GTGTTTTGAG ACTATAAATA TCCCTTGGAG |
| 2051 | AAAAGCCTTG TTTGGGCCCC CCCTCGAGGT CGACGGTATC GATAAGCTTG |
| 2101 | ATATCGAATT CCTGCAGCCC GGGGGATCC |

TABLE 3
GFAP SmaI Promoter (Length 2169)

VERSION pDRAW 1.0 beta
DNAname New DNA entry
IScircular NO
Sequence . . .

| | |
|---|---|
| 1 | AGATCTGAGC TCCCACCTCC CTCTCTGTGC TGGGACTCAC AGAGGGAGAC |
| 51 | CTCAGGAGGC AGTCTGTCCA TCACATGTCC AAATGCAGAG CATACCCTGG |
| 101 | GCTGGGCGCA GTGGCGCACA ACTGTAATTC CAGCACTTTG GGAGGCTGAT |
| 151 | GTGGAAGGAT CACTTGAGCC CAGAAGTTCT AGACCAGCCT GGGCAACATG |
| 201 | GCAAGACCCT ATCTCTACAA AAAAAGTTAA AAAATCAGCC ACGTGTGGTG |
| 251 | ACACACACCT GTAGTCCCAG CTATTCAGGA GGCTGAGGTG AGGGGATCAC |
| 301 | TTAAGGCTGG GAGGTTGAGG CTGCAGTGAG TCGTGGTTGC GCCACTGCAC |
| 351 | TCCAGCCTGG GCAACAGTGA GACCCTGTCT CAAAAGACAA AAAAAAAAAA |
| 401 | AAAAAAAAAA AGAACATATC CTGGTGTGGA GTAGGGGACG CTGCTCTGAC |
| 451 | AGAGGCTCGG GGGCCTGAGC TGGCTCTGTG AGCTGGGGAG GAGGCAGACA |
| 501 | GCCAGGCCTT GTCTGCAAGC AGACCTGGCA GCATTGGGCT GGCCGCCCCC |
| 551 | CAGGGCCTCC TCTTCATGCC CAGTGAATGA CTCACCTTGG CACAGACACA |
| 601 | ATGTTCGGGG TGGGCACAGT GCCTGCTTCC CGCCGCACCC CAGCCCCCCT |
| 651 | CAAATGCCTT CCGAGAAGCC CATTGAGCAG GGGGCTTGCA TTGCACCCCA |
| 701 | GCCTGACAGC CTGGCATCTT GGGATAAAAG CAGCACAGCC CCCTAGGGGC |
| 751 | TGCCCTTGCT GTGTGGCGCC ACCGGCGGTG GAGAACAAGG CTCTATTCAG |
| 801 | CCTGTGCCCA GGAAAGGGGA TCAGGGGATG CCCAGGCATG GACAGTGGGT |
| 851 | GGCAGGGGGG GAGAGGAGGG CTGTCTGCTT CCCAGAAGTC CAAGGACACA |
| 901 | AATGGGTGAG GGGACTGGGC AGGGTTCTGA CCCTGTGGGA CCAGAGTGGA |
| 951 | GGGCGTAGAT GGACCTGAAG TCTCCAGGGA CAACAGGGCC CAGGTCTCAG |
| 1001 | GCTCCTAGTT GGGCCCAGTG GCTCCAGCGT TTCCAAACCC ATCCATCCCC |
| 1051 | AGAGGTTCTT CCCATCTCTC CAGGCTGATG TGTGGGAACT CGAGGAAATA |
| 1101 | AATCTCCAGT GGGAGACGGA GGGGTGGCCA GGGAAACGGG GCGCTGCAGG |
| 1151 | AATAAAGACG AGCCAGCACA GCCAGCTCAT GTGTAACGGC TTTGTGGAGC |
| 1201 | TGTCAAGGCC TGGTCTCTGG GAGAGAGGCA CAGGGAGGCC AGACAAGGAA |
| 1251 | GGGGTGACCT GGAGGGACAG ATCCAGGGGC TAAAGTCCTG ATAAGGCAAG |
| 1301 | AGAGTGCCGG CCCCCTCTTG CCCTATCAGG ACCTCCACTG CCACATAGAG |
| 1351 | GCCATGATTG ACCCTTAGAC AAAGGGCTGG TGTCCAATCC CAGCCCCCAG |
| 1401 | CCCCAGAACT CCAGGGAATG AATGGGCAGA GAGCAGGAAT GTGGGACATC |
| 1451 | TGTGTTCAAG GGAAGGACTC CAGGAGTCTG CTGGGAATGA GGCCTAGTAG |
| 1501 | GAAATGAGGT GGCCCTTGAG GGTACAGAAC AGGTTCATTC TTCGCCAAAT |
| 1551 | TCCCAGCACC TTGCAGGCAC TTACAGCTGA GTGAGATAAT GCCTGGGTTA |
| 1601 | TGAAATCAAA AAGTTGGAAA GCAGGTCAGA GGTCATCTGG TACAGCCCTT |
| 1651 | CCTTCCCTTT TTTTTTTTTT TTTTTTGTGA GACAAGGTCT CTCTCTGTTG |
| 1701 | CCCAGGCTGG AGTGGCGCAA ACACAGCTCA CTGCAGCCTC AACCTACTGG |
| 1751 | GCTCAAGCAA TCCTCCAGCC TCAGCCTCCC AAAGTGCTGG GATTACAAGC |
| 1801 | ATGAGCCACC CCACTCAGCC CTTTCCTTCC TTTTTAATTG ATGCATAATA |
| 1851 | ATTGTAAGTA TTCATCATGG TCCAACCAAC CCTTTCTTGA CCCACCTTCC |
| 1901 | TAGAGAGAGG GTCCTCTTGC TTCAGCGGTC AGGGCCCCAG ACCCATGGTC |
| 1951 | TGGCTCCAGG TACCACCTGC CTCATGCAGG AGTTGGCGTG CCCAGGAAGC |
| 2001 | TCTGCCTCTG GGCACAGTGA CCTCAGTGGG GTGAGGGGAG CTCTCCCCAT |
| 2051 | AGCTGGGCTG CGGCCCAACC CCACCCCCTC AGGCTATGCC AGGGGGTGTT |
| 2101 | GCCAGGGGCA CCCTAAAAAA CAGCACAAAA GGAAACTCAC CCTAACTGTA |
| 2151 | AAGTAATTGT GTGTTTTGAG ACTATAAATA TCCCTTGGAG AAAAGCCTTG |
| 2201 | TTTGGGCCCC CCCTCGAGGT CGACGGTATC GATAAGCTTG ATATCGAATT |
| 2251 | CCTGCAGCCC GGGGGATCC |

TABLE 4
gpGFP-HcRed1 (Length 6710 bp)

VERSION pDRAW 1.0 beta
DNAname peGFP-HcRed1
IScircular YES

| | | | | |
|---|---|---|---|---|
| Element | CMVie | 1 | 1040 | 1 |
| −1 | | | | |
| Element | eGFP | 1070 | 1794 | 0 |
| −1 | | | | |
| Element | CMVie | 1950 | 3040 | 1 |
| −1 | | | | |
| Element | HcRed1 | 3050 | 3798 | 0 |
| −1 | | | | |

Sequence . . .

| | |
|---|---|
| 1 | TCAATATTGG CCATTAGCCA TATTATTCAT TGGTTATATA GCATAAATCA |
| 51 | ATATTGGCTA TTGGCCATTG CATACGTTGT ATCTATATCA TAATATGTAC |
| 101 | ATTTATATTG GCTCATGTCC AATATGACCG CCATGTTGGC ATTGATTATT |
| 151 | GACTAGTTAT TAATAGTAAT CAATTACGGG GTCATTAGTT CATAGCCCAT |
| 201 | ATATGGAGTT CCGCGTTACA TAACTTACGG TAAATGGCCC GCCTGGCTGA |
| 251 | CCGCCCAACG ACCCCCGCCC ATTGACGTCA ATAATGACGT ATGTTCCCAT |
| 301 | AGTAACGCCA ATAGGGACTT TCCATTGACG TCAATGGGTG GAGTATTTAC |
| 351 | GGTAAACTGC CCACTTGGCA GTACATCAAG TGTATCATAT GCCAAGTCCG |
| 401 | CCCCCTATTG ACGTCAATGA CGGTAAATGG CCCGCCTGGC ATTATGCCCA |
| 451 | GTACATGACC TTACGGGACT TTCCTACTTG GCAGTACATC TACGTATTAG |
| 501 | TCATCGCTAT TACCATGGTG ATGCGGTTTT GGCAGTACAC CAATGGGCGT |
| 551 | GGATAGCGGT TTGACTCACG GGGATTTCCA AGTCTCCACC CCATTGACGT |
| 601 | CAATGGGAGT TTGTTTTGGC ACCAAAATCA ACGGGACTTT CCAAAATGTC |
| 651 | GTAATAACCC CGCCCCGTTG ACGCAAATGG GCGGTAGGCG TGTACGGTGG |
| 701 | GAGGTCTATA TAAGCAGAGC TCGTTTAGTG AACCGTCAGA TCACTAGAAG |
| 751 | CTTTATTGCG GTAGTTTATC ACAGTTAAAT TGCTAACGCA GTCAGTGCTT |
| 801 | CTGACACAAC AGTCTCGAAC TTAAGCTGCA GAAGTTGGTC GTGAGGCACT |
| 851 | GGGCAGGTAA GTATCAAGGT TACAAGACAG GTTTAAGGAG ACCAATAGAA |
| 901 | ACTGGGCTTG TCGAGACAGA GAAGACTCTT GCGTTTCTGA TAGGCACCTA |
| 951 | TTGGTCTTAC TGACATCCAC TTTGCCTTTC TCTCCACAGG TGTCCACTCC |
| 1001 | CAGTTCAATT ACAGCTCTTA AGGCTAGAGT ACTTAATACG ACTCACTATA |
| 1051 | GGCTAGGGGT ACCGGTCGCC ACCATGGTGA GCAAGGGCGA GGAGCTGTTC |
| 1101 | ACCGGGGTGG TGCCCATCCT GGTCGAGCTG GACGGCGACG TAAACGGCCA |
| 1151 | CAAGTTCAGC GTGTCCGGCG AGGGCGAGGG CGATGCCACC TACGGCAAGC |
| 1201 | TGACCCTGAA GTTCATCTGC ACCACCGGCA AGCTGCCCGT GCCCTGGCCC |
| 1251 | ACCCTCGTGA CCACCCTGAC CTACGGCGTG CAGTGCTTCA GCCGCTACCC |
| 1301 | CGACCACATG AAGCAGCACG ACTTCTTCAA GTCCGCCATG CCCGAAGGCT |
| 1351 | ACGTCCAGGA GCGCACCATC TTCTTCAAGG ACGACGGCAA CTACAAGACC |
| 1401 | CGCGCCGAGG TGAAGTTCGA GGGCGACACC CTGGTGAACC GCATCGAGCT |
| 1451 | GAAGGGCATC GACTTCAAGG AGGACGGCAA CATCCTGGGG CACAAGCTGG |
| 1501 | AGTACAACTA CAACAGCCAC AACGTCTATA TCATGGCCGA CAAGCAGAAG |
| 1551 | AACGGCATCA AGGTGAACTT CAAGATCCGC CACAACATCG AGGACGGCAG |
| 1601 | CGTGCAGCTC GCCGACCACT ACCAGCAGAA CACCCCCATC GGCGACGGCC |
| 1651 | CCGTGCTGCT GCCCGACAAC CACTACCTGA GCACCCAGTC CGCCCTGAGC |
| 1701 | AAAGACCCCA ACGAGAAGCG CGATCACATG GTCCTGCTGG AGTTCGTGAC |
| 1751 | CGCCGCCGGG ATCACTCTCG GCATGGACGA GCTGTACAAG TAAAGCGGCC |
| 1801 | GCTTCGAGCA GACATGATAA GATACATTGA TGAGTTTGGA CAAACCACAA |
| 1851 | CTAGAATGCA GTGAAAAAAA TGCTTTATTT GTGAAATTTG TGATGCTATT |
| 1901 | GCTTTATTTG TAACCATTAT AAGCTGCAAT AAACAAGTTA ACAACAACAA |
| 1951 | TTGCATTCAT TTTATGTTTC AGGTTCAGGG GGAGATGTGG GAGGTTTTTT |
| 2001 | AAAGCAAGTA AAACCTCTAC AAATGTGGTA AAATCGATAA GGATCTTCAA |
| 2051 | TATTGGCCAT TAGCCATATT ATTCATTGGT TATATAGCAT AAATCAATAT |
| 2101 | TGGCTATTGG CCATTGCATA CGTTGTATCT ATATCATAAT ATGTACATTT |
| 2151 | ATATTGGCTC ATGTCCAATA TGACCGCCAT GTTGGCATTG ATTATTGACT |
| 2201 | AGTTATTAAT AGTAATCAAT TACGGGGTCA TTAGTTCATA GCCCATATAT |
| 2251 | GGAGTTCCGC GTTACATAAC TTACGGTAAA TGGCCCGCCT GGCTGACCGC |
| 2301 | CCAACGACCC CCGCCCATTG ACGTCAATAA TGACGTATGT TCCCATAGTA |
| 2351 | ACGCCAATAG GGACTTTCCA TTGACGTCAA TGGGTGGAGT ATTTACGGTA |
| 2401 | AACTGCCCAC TTGGCAGTAC ATCAAGTGTA TCATATGCCA AGTCCGCCCC |
| 2451 | CTATTGACGT CAATGACGGT AAATGGCCCG CCTGGCATTA TGCCCAGTAC |
| 2501 | ATGACCTTAC GGGACTTTCC TACTTGGCAG TACATCTACG TATTAGTCAT |
| 2551 | CGCTATTACC ATGGTGATGC GGTTTTGGCA GTACACCAAT GGGCGTGGAT |
| 2601 | AGCGGTTTGA CTCACGGGGA TTTCCAAGTC TCCACCCCAT TGACGTCAAT |
| 2651 | GGGAGTTTGT TTTGGCACCA AAATCAACGG GACTTTCCAA AATGTCGTAA |
| 2701 | TAACCCCGCC CCGTTGACGC AAATGGGCGG TAGGCGTGTA CGGTGGGAGG |
| 2751 | TCTATATAAG CAGAGCTCGT TTAGTGAACC GTCAGATCAC TAGAAGCTTT |
| 2801 | ATTGCGGTAG TTTATCACAG TTAAATTGCT AACGCAGTCA GTGCTTCTGA |
| 2851 | CACAACAGTC TCGAACTTAA GCTGCAGAAG TTGGTCGTGA GGCACTGGGC |
| 2901 | AGGTAAGTAT CAAGGTTACA AGACAGGTTT AAGGAGACCA ATAGAAACTG |
| 2951 | GGCTTGTCGA GACAGAGAAG ACTCTTGCGT TTCTGATAGG CACCTATTGG |
| 3001 | TCTTACTGAC ATCCACTTTG CCTTTCTCTC CACAGGTGTC CACTCCCAGT |
| 3051 | TCAATTACAG CTCTTAAGGC TAGAGTACTT AATACGACTC ACTATAGGCT |
| 3101 | AGTCGCCACC ATGGTGAGCG GCCTGCTGAA GGAGAGTATG CGCATCAAGA |
| 3151 | TGTACATGGA GGGCACCGTG AACGGCCACT ACTTCAAGTG CGAGGGCGAG |
| 3201 | GGCGACGGCA ACCCCTTCGC CGGCACCCAG AGCATGAGAA TCCACGTGAC |
| 3251 | CGAGGGCGCC CCCCTGCCCT TCGCCTTCGA CATCCTGGCC CCCTGCTGCG |
| 3301 | AGTACGGCAG CAGGACCTTC GTGCACCACA CCGCCGAGAT CCCCGACTTC |
| 3351 | TTCAAGCAGA GCTTCCCCGA GGGCTTCACC TGGGAGAGAA CCACCACCTA |
| 3401 | CGAGGACGGC GGCATCCTGA CCGCCCACCA GGACACCAGC CTGGAGGGCA |
| 3451 | ACTGCCTGAT CTACAAGGTG AAGGTGCACG GCACCAACTT CCCCGCCGAC |
| 3501 | GGCCCCGTGA TGAAGAACAA GAGCGGCGGC TGGGAGCCCA GCACCGAGGT |
| 3551 | GGTGTACCCC GAGAACGGCG TGCTGTGCGG CCGGAACGTG ATGGCCCTGA |
| 3601 | AGGTGGGCGA CCGGCACCTG ATCTGCCACC ACTACACCAG CTACCGGAGC |
| 3651 | AAGAAGGCCG TGCGCGCCCT GACCATGCCC GGCTTCCACT TCACCGACAT |
| 3701 | CCGGCTCCAG ATGCTGCGGA AGAAGAAGGA CGAGTACTTC GAGCTGTACG |
| 3751 | AGGCCAGCGT GGCCCGGTAC AGCGACCTGC CCGAGAAGGC CAACTGAAGC |
| 3801 | GGCCGCTTCG AGCAGACATG ATAAGATACA TTGATGAGTT TGGACAAACC |
| 3851 | ACAACTAGAA TGCAGTGAAA AAAATGCTTT ATTTGTGAAA TTTGTGATGC |
| 3901 | TATTGCTTTA TTTGTAACCA TTATAAGCTG CAATAAACAA GTTAACAACA |
| 3951 | ACAATTGCAT TCATTTTATG TTTCAGGTTC AGGGGGAGAT GTGGGAGGTT |
| 4001 | TTTTAAAGCA AGTAAAACCT CTACAAATGT GGTAAAATCG ATAAGGATCC |
| 4051 | GGGCTGGCGT AATAGCGAAG AGGCCCGCAC CGATCGCCCT TCCCAACAGT |
| 4101 | TGCGCAGCCT GAATGGCGAA TGGACGCGCC CTGTAGCGGC GCATTAAGCG |
| 4151 | CGGCGGGTGT GGTGGTTACG CGCAGCGTGA CCGCTACACT TGCCAGCGCC |
| 4201 | CTAGCGCCCG CTCCTTTCGC TTTCTTCCCT TCCTTTCTCG CCACGTTCGC |
| 4251 | CGGCTTTCCC CGTCAAGCTC TAAATCGGGG GCTCCCTTTA GGGTTCCGAT |
| 4301 | TTAGAGCTTT ACGGCACCTC GACCGCAAAA AACTTGATTT GGGTGATGGT |
| 4351 | TCACGTAGTG GGCCATCGCC CTGATAGACG GTTTTTCGCC CTTTGACGTT |
| 4401 | GGAGTCCACG TTCTTTAATA GTGGACTCTT GTTCCAAACT GGAACAACAC |
| 4451 | TCAACCCTAT CTCGGTCTAT TCTTTTGATT TATAAGGGAT TTTGCCGATT |
| 4501 | TCGGCCTATT GGTTAAAAAA TGAGCTGATT TAACAAATAT TTAACGCGAA |
| 4551 | TTTTAACAAA ATATTAACGT TTACAATTTC GCCTGATGCG GTATTTTCTC |
| 4601 | CTTACGCATC TGTGCGGTAT TTCACACCGC ATATGGTGCA CTCTCAGTAC |
| 4651 | AATCTGCTCT GATGCCGCAT AGTTAAGCCA GCCCCGACAC CCGCCAACAC |
| 4701 | CCGCTGACGC GCCCTGACGG GCTTGTCTGC TCCCGGCATC CGCTTACAGA |
| 4751 | CAAGCTGTGA CCGTCTCCGG GAGCTGCATG TGTCAGAGGT TTTCACCGTC |
| 4801 | ATCACCGAAA CGCGCGAGAC GAAAGGGCCT CGTGATACGC CTATTTTTAT |
| 4851 | AGGTTAATGT CATGATAATA ATGGTTTCTT AGACGTCAGG TGGCACTTTT |
| 4901 | CGGGGAAATG TGCGCGGAAC CCCTATTTGT TTATTTTTCT AAATACATTC |
| 4951 | AAATATGTAT CCGCTCATGA GACAATAACC CTGATAAATG CTTCAATAAT |
| 5001 | ATTGAAAAAG GAAGAGTATG AGTATTCAAC ATTTCCGTGT CGCCCTTATT |
| 5051 | CCCTTTTTTG CGGCATTTTG CCTTCCTGTT TTTGCTCACC CAGAAACGCT |
| 5101 | GGTGAAAGTA AAAGATGCTG AAGATCAGTT GGGTGCACGA GTGGGTTACA |
| 5151 | TCGAACTGGA TCTCAACAGC GGTAAGATCC TTGAGAGTTT TCGCCCCGAA |
| 5201 | GAACGTTTTC CAATGATGAG CACTTTTAAA GTTCTGCTAT GTGGCGCGGT |
| 5251 | ATTATCCCGT ATTGACGCCG GGCAAGAGCA ACTCGGTCGC CGCATACACT |
| 5301 | ATTCTCAGAA TGACTTGGTT GAGTACTCAC CAGTCACAGA AAAGCATCTT |
| 5351 | ACGGATGGCA TGACAGTAAG AGAATTATGC AGTGCTGCCA TAACCATGAG |
| 5401 | TGATAACACT GCGGCCAACT TACTTCTGAC AACGATCGGA GGACCGAAGG |
| 5451 | AGCTAACCGC TTTTTTGCAC AACATGGGGG ATCATGTAAC TCGCCTTGAT |
| 5501 | CGTTGGGAAC CGGAGCTGAA TGAAGCCATA CCAAACGACG AGCGTGACAC |
| 5551 | CACGATGCCT GTAGCAATGG CAACAACGTT GCGCAAACTA TTAACTGGCG |
| 5601 | AACTACTTAC TCTAGCTTCC CGGCAACAAT TAATAGACTG GATGGAGGCG |
| 5651 | GATAAAGTTG CAGGACCACT TCTGCGCTCG GCCCTTCCGG CTGGCTGGTT |
| 5701 | TATTGCTGAT AAATCTGGAG CCGGTGAGCG TGGGTCTCGC GGTATCATTG |
| 5751 | CAGCACTGGG GCCAGATGGT AAGCCCTCCC GTATCGTAGT TATCTACACG |
| 5801 | ACGGGGAGTC AGGCAACTAT GGATGAACGA AATAGACAGA TCGCTGAGAT |
| 5851 | AGGTGCCTCA CTGATTAAGC ATTGGTAACT GTCAGACCAA GTTTACTCAT |
| 5901 | ATATACTTTA GATTGATTTA AAACTTCATT TTTAATTTAA AAGGATCTAG |
| 5951 | GTGAAGATCC TTTTTGATAA TCTCATGACC AAAATCCCTT AACGTGAGTT |
| 6001 | TTCGTTCCAC TGAGCGTCAG ACCCCGTAGA AAAGATCAAA GGATCTTCTT |
| 6051 | GAGATCCTTT TTTTCTGCGC GTAATCTGCT GCTTGCAAAC AAAAAAACCA |
| 6101 | CCGCTACCAG CGGTGGTTTG TTTGCCGGAT CAAGAGCTAC CAACTCTTTT |
| 6151 | TCCGAAGGTA ACTGGCTTCA GCAGAGCGCA GATACCAAAT ACTGTCCTTC |
| 6201 | TAGTGTAGCC GTAGTTAGGC CACCACTTCA AGAACTCTGT AGCACCGCCT |
| 6251 | ACATACCTCG CTCTGCTAAT CCTGTTACCA GTGGCTGCTG CCAGTGGCGA |
| 6301 | TAAGTCGTGT CTTACCGGGT TGGACTCAAG ACGATAGTTA CCGGATAAGG |
| 6351 | CGCAGCGGTC GGGCTGAACG GGGGGTTCGT GCACACAGCC CAGCTTGGAG |
| 6401 | CGAACGACCT ACACCGAACT GAGATACCTA CAGCGTGAGC TATGAGAAAG |
| 6451 | CGCCACGCTT CCCGAAGGGA GAAAGGCGGA CAGGTATCCG GTAAGCGGCA |
| 6501 | GGGTCGGAAC AGGAGAGCGC ACGAGGGAGC TTCCAGGGGG AAACGCCTGG |
| 6551 | TATCTTTATA GTCCTGTCGG GTTTCGCCAC CTCTGACTTG AGCGTCGATT |
| 6601 | TTTGTGATGC TCGTCAGGGG GGCGGAGCCT ATGGAAAAAC GCCAGCAACG |
| 6651 | CGGCCTTTTT ACGGTTCCTG GCCTTTTGCT GGCCTTTTGC TCACATGGCT |
| 6701 | CGACAGATCT |

TABLE 5
pGFA2-LacZ (Length 8569 bp)

VERSION pDRAW 1.0 beta
DNAname pGFA2-LacZ from Brenner
IScircular YES
Sequence . . .

| | |
|---|---|
| 1 | GCGCCCAATA CGCAAACCGC CTCTCCCCGC GCGTTGGCCG ATTCATTAAT |
| 51 | GCAGCTGGCA CGACAGGTTT CCCGACTGGA AAGCGGGCAG TGAGCGCAAC |
| 101 | GCAATTAATG TGAGTTAGCT CACTCATTAG GCACCCCAGG CTTTACACTT |
| 151 | TATGCTTCCG GCTCGTATGT TGTGTGGAAT TGTGAGCGGA TAACAATTTC |
| 201 | ACACAGGAAA CAGCTATGAC ATGATTACGA ATTCGAGCTC GGTACCAGAT |
| 251 | CTGAGCTCCC ACCTCCCTCT CTGTGCTGGG ACTCACAGAG GGAGACCTCA |
| 301 | GGAGGCAGTC TGTCCATCAC ATGTCCAAAT GCAGAGCATA CCCTGGGCTG |
| 351 | GGCGCAGTGG CGCACAACTG TAATTCCAGC ACTTTGGGAG GCTGATGTGG |
| 401 | AAGGATCACT TGAGCCCAGA AGTTCTAGAC CAGCCTGGGC AACATGGCAA |
| 451 | GACCCTATCT CTACAAAAAA AGTTAAAAAA TCAGCCACGT GTGGTGACAC |
| 501 | ACACCTGTAG TCCCAGCTAT TCAGGAGGCT GAGGTGAGGG GATCACTTAA |
| 551 | GGCTGGGAGG TTGAGGCTGC AGTGAGTCGT GGTTGCGCCA CTGCACTCCA |
| 601 | GCCTGGGCAA CAGTGAGACC CTGTCTCAAA AGACAAAAAA AAAAAAAAAA |
| 651 | AAAAAAAGAA CATATCCTGG TGTGGAGTAG GGGACGCTGC TCTGACAGAG |
| 701 | GCTCGGGGGC CTGAGCTGGC TCTGTGAGCT GGGGAGGAGG CAGACAGCCA |
| 751 | GGCCTTGTCT GCAAGCAGAC CTGGCAGCAT TGGGCTGGCC GCCCCCCAGG |
| 801 | GCCTCCTCTT CATGCCCAGT GAATGACTCA CCTTGGCACA GACACAATGT |
| 851 | TCGGGGTGGG CACAGTGCCT GCTTCCCGCC GCACCCCAGC CCCCCTCAAA |
| 901 | TGCCTTCCGA GAAGCCCATT GAGCAGGGGG CTTGCATTGC ACCCCAGCCT |
| 951 | GACAGCCTGG CATCTTGGGA TAAAAGCAGC ACAGCCCCCT AGGGGCTGCC |
| 1001 | CTTGCTGTGT GGCGCCACCG GCGGTGGAGA ACAAGGCTCT ATTCAGCCTG |
| 1051 | TGCCCAGGAA AGGGGATCAG GGGATGCCCA GGCATGGACA GTGGGTGGCA |
| 1101 | GGGGGGGAGA GGAGGGCTGT CTGCTTCCCA GAAGTCCAAG GACACAAATG |
| 1151 | GGTGAGGGGA CTGGGCAGGG TTCTGACCCT GTGGGACCAG AGTGGAGGGC |
| 1201 | GTAGATGGAC CTGAAGTCTC CAGGGACAAC AGGGCCCAGG TCTCAGGCTC |
| 1251 | CTAGTTGGGC CCAGTGGCTC CAGCGTTTCC AAACCCATCC ATCCCCAGAG |
| 1301 | GTTCTTCCCA TCTCTCCAGG CTGATGTGTG GGAACTCGAG GAAATAAATC |
| 1351 | TCCAGTGGGA GACGGAGGGG TGGCCAGGGA AACGGGGCGC TGCAGGAATA |
| 1401 | AAGACGAGCC AGCACAGCCA GCTCATGTGT AACGGCTTTG TGGAGCTGTC |
| 1451 | AAGGCCTGGT CTCTGGGAGA GAGGCACAGG GAGGCCAGAC AAGGAAGGGG |
| 1501 | TGACCTGGAG GGACAGATCC AGGGGCTAAA GTCCTGATAA GGCAAGAGAG |
| 1551 | TGCCGGCCCC CTCTTGCCCT ATCAGGACCT CCACTGCCAC ATAGAGGCCA |
| 1601 | TGATTGACCC TTAGACAAAG GGCTGGTGTC CAATCCCAGC CCCCAGCCCC |
| 1651 | AGAACTCCAG GGAATGAATG GGCAGAGAGC AGGAATGTGG GACATCTGTG |
| 1701 | TTCAAGGGAA GGACTCCAGG AGTCTGCTGG GAATGAGGCC TAGTAGGAAA |
| 1751 | TGAGGTGGCC CTTGAGGGTA CAGAACAGGT TCATTCTTCG CCAAATTCCC |
| 1801 | AGCACCTTGC AGGCACTTAC AGCTGAGTGA GATAATGCCT GGGTTATGAA |
| 1851 | ATCAAAAAGT TGGAAAGCAG GTCAGAGGTC ATCTGGTACA GCCCTTCCTT |
| 1901 | CCCTTTTTTT TTTTTTTTTT TTGTGAGACA AGGTCTCTCT CTGTTGCCCA |
| 1951 | GGCTGGAGTG GCGCAAACAC AGCTCACTGC AGCCTCAACC TACTGGGCTC |
| 2001 | AAGCAATCCT CCAGCCTCAG CCTCCCAAAG TGCTGGGATT ACAAGCATGA |
| 2051 | GCCACCCCAC TCAGCCCTTT CCTTCCTTTT TAATTGATGC ATAATAATTG |
| 2101 | TAAGTATTCA TCATGGTCCA ACCAACCCTT TCTTGACCCA CCTTCCTAGA |
| 2151 | GAGAGGGTCC TCTTGCTTCA GCGGTCAGGG CCCCAGACCC ATGGTCTGGC |
| 2201 | TCCAGGTACC ACCTGCCTCA TGCAGGAGTT GGCGTGCCCA GGAAGCTCTG |
| 2251 | CCTCTGGGCA CAGTGACCTC AGTGGGGTGA GGGGAGCTCT CCCCATAGCT |
| 2301 | GGGCTGCGGC CCAACCCCAC CCCCTCAGGC TATGCCAGGG GGTGTTGCCA |
| 2351 | GGGGCACCCG GGCATCGCCA GTCTAGCCCA CTCCTTCATA AAGCCCTCGC |
| 2401 | ATCCCAGGAG CGAGCAGAGC CAGAGCAGGT TGGAGAGGAG ACGCATCACC |
| 2451 | TCCGCTGCTC GCGGGGATCC TCTAGAGTCG ACGGATCCGG GGAATTCCCC |
| 2501 | AGTCTCAGGA TCCACCATGG GGGATCCCGT CGTTTTACAA CGTCGTGACT |
| 2551 | GGGAAAACCC TGGCGTTACC CAACTTAATC GCCTTGCAGC ACATCCCCCT |
| 2601 | TTCGCCAGCT GGCGTAATAG CGAAGAGGCC CGCACCGATC GCCCTTCCCA |
| 2651 | ACAGTTGCGC AGCCTGAATG GCGAATGGCG CTTTGCCTGG TTTCCGGCAC |
| 2701 | CAGAAGCGGT GCCGGAAAGC TGGCTGGAGT GCGATCTTCC TGAGGCCGAT |
| 2751 | ACTGTCGTCG TCCCCTCAAA CTGGCAGATG CACGGTTACG ATGCGCCCAT |
| 2801 | CTACACCAAC GTAACCTATC CCATTACGGT CAATCCGCCG TTTGTTCCCA |
| 2851 | CGGAGAATCC GACGGGTTGT TACTCGCTCA CATTTAATGT TGATGAAAGC |
| 2901 | TGGCTACAGG AAGGCCAGAC GCGAATTATT TTTGATGGCG TTAACTCGGC |
| 2951 | GTTTCATCTG TGGTGCAACG GGCGCTGGGT CGGTTACGGC CAGGACAGTC |
| 3001 | GTTTGCCGTC TGAATTTGAC CTGAGCGCAT TTTTACGCGC CGGAGAAAAC |
| 3051 | CGCCTCGCGG TGATGGTGCT GCGTTGGAGT GACGGCAGTT ATCTGGAAGA |
| 3101 | TCAGGATATG TGGCGGATGA GCGGCATTTT CCGTGACGTC TCGTTGCTGC |
| 3151 | ATAAACCGAC TACACAAATC AGCGATTTCC ATGTTGCCAC TCGCTTTAAT |
| 3201 | GATGATTTCA GCCGCGCTGT ACTGGAGGCT GAAGTTCAGA TGTGCGGCGA |
| 3251 | GTTGCGTGAC TACCTACGGG TAACAGTTTC TTTATGGCAG GGTGAAACGC |
| 3301 | AGGTCGCCAG CGGCACCGCG CCTTTCGGCG GTGAAATTAT CGATGAGCGT |
| 3351 | GGTGGTTATG CCGATCGCGT CACACTACGT CTGAACGTCG AAAACCCGAA |
| 3401 | ACTGTGGAGC GCCGAAATCC CGAATCTCTA TCGTGCGGTG GTTGAACTGC |
| 3451 | ACACCGCCGA CGGCACGCTG ATTGAAGCAG AAGCCTGCGA TGTCGGTTTC |
| 3501 | CGCGAGGTGC GGATTGAAAA TGGTCTGCTG CTGCTGAACG GCAAGCCGTT |
| 3551 | GCTGATTCGA GGCGTTAACC GTCACGAGCA TCATCCTCTG CATGGTCAGG |
| 3601 | TCATGGATGA GCAGACGATG GTGCAGGATA TCCTGCTGAT GAAGCAGAAC |
| 3651 | AACTTTAACG CCGTGCGCTG TTCGCATTAT CCGAACCATC CGCTGTGGTA |
| 3701 | CACGCTGTGC GACCGCTACG GCCTGTATGT GGTGGATGAA GCCAATATTG |
| 3751 | AAACCCACGG CATGGTGCCA ATGAATCGTC TGACCGATGA TCCGCGCTGG |
| 3801 | CTACCGGCGA TGAGCGAACG CGTAACGCGA ATGGTGCAGC GCGATCGTAA |
| 3851 | TCACCCGAGT GTGATCATCT GGTCGCTGGG GAATGAATCA GGCCACGGCG |
| 3901 | CTAATCACGA CGCGCTGTAT CGCTGGATCA AATCTGTCGA TCCTTCCCGC |
| 3951 | CCGGTGCAGT ATGAAGGCGG CGGAGCCGAC ACCACGGCCA CCGATATTAT |
| 4001 | TTGCCCGATG TACGCGCGCG TGGATGAAGA CCAGCCCTTC CCGGCTGTGC |
| 4051 | CGAAATGGTC CATCAAAAAA TGGCTTTCGC TACCTGGAGA GACGCGCCCG |
| 4101 | CTGATCCTTT GCGAATACGC CCACGCGATG GGTAACAGTC TTGGCGGTTT |
| 4151 | CGCTAAATAC TGGCAGGCGT TTCGTCAGTA TCCCCGTTTA CAGGGCGGCT |
| 4201 | TCGTCTGGGA CTGGGTGGAT CAGTCGCTGA TTAAATATGA TGAAAACGGC |
| 4251 | AACCCGTGGT CGGCTTACGG CGGTGATTTT GGCGATACGC CGAACGATCG |
| 4301 | CCAGTTCTGT ATGAACGGTC TGGTCTTTGC CGACCGCACG CCGCATCCAG |
| 4351 | CGCTGACGGA AGCAAAACAC CAGCAGCAGT TTTTCCAGTT CCGTTTATCC |
| 4401 | GGGCAAACCA TCGAAGTGAC CAGCGAATAC CTGTTCCGTC ATAGCGATAA |
| 4451 | CGAGCTCCTG CACTGGATGG TGGCGCTGGA TGGTAAGCCG CTGGCAAGCG |
| 4501 | GTGAAGTGCC TCTGGATGTC GCTCCACAAG GTAAACAGTT GATTGAACTG |
| 4551 | CCTGAACTAC CGCAGCCGGA GAGCGCCGGG CAACTCTGGC TCACAGTACG |
| 4601 | CGTAGTGCAA CCGAACGCGA CCGCATGGTC AGAAGCCGGG CACATCAGCG |
| 4651 | CCTGGCAGCA GTGGCGTCTG GCGGAAAACC TCAGTGTGAC GCTCCCCGCC |
| 4701 | GCGTCCCACG CCATCCCGCA TCTGACCACC AGCGAAATGG ATTTTTGCAT |
| 4751 | CGAGCTGGGT AATAAGCGTT GGCAATTTAA CCGCCAGTCA GGCTTTCTTT |
| 4801 | CACAGATGTG GATTGGCGAT AAAAAACAAC TGCTGACGCC GCTGCGCGAT |
| 4851 | CAGTTCACCC GTGCACCGCT GGATAACGAC ATTGGCGTAA GTGAAGCGAC |
| 4901 | CCGCATTGAC CCTAACGCCT GGGTCGAACG CTGGAAGGCG GCGGGCCATT |
| 4951 | ACCAGGCCGA AGCAGCGTTG TTGCAGTGCA CGGCAGATAC ACTTGCTGAT |
| 5001 | GCGGTGCTGA TTACGACCGC TCACGCGTGG CAGCATCAGG GGAAAACCTT |
| 5051 | ATTTATCAGC CGGAAAACCT ACCGGATTGA TGGTAGTGGT CAAATGGCGA |
| 5101 | TTACCGTTGA TGTTGAAGTG GCGAGCGATA CACCGCATCC GGCGCGGATT |
| 5151 | GGCCTGAACT GCCAGCTGGC GCAGGTAGCA GAGCGGGTAA ACTGGCTCGG |
| 5201 | ATTAGGGCCG CAAGAAAACT ATCCCGACCG CCTTACTGCC GCCTGTTTTG |
| 5251 | ACCGCTGGGA TCTGCCATTG TCAGACATGT ATACCCCGTA CGTCTTCCCG |
| 5301 | AGCGAAAACG GTCTGCGCTG CGGGACGCGC GAATTGAATT ATGGCCCACA |
| 5351 | CCAGTGGCGC GGCGACTTCC AGTTCAACAT CAGCCGCTAC AGTCAACAGC |
| 5401 | AACTGATGGA AACCAGCCAT CGCCATCTGC TGCACGCGGA AGAAGGCACA |
| 5451 | TGGCTGAATA TCGACGGTTT CCATATGGGG ATTGGTGGCG ACGACTCCTG |
| 5501 | GAGCCCGTCA GTATCGGCGG AATTCCAGCT GAGCGCCGGT CGCTACCATT |
| 5551 | ACCAGTTGGT CTGGTGTCAA AAATAATAAT AACCGGGCAG GGGGGATCCG |
| 5601 | CAGATCCCGG CCAGATACCG ATGCTGCCGC AGCAAAAGCA GGAGCAGATG |
| 5651 | CCGCCGTCGC AGGCGAAGAT GTCGCAGACG GAGGAGGCGA TGCTGCCGGC |
| 5701 | GGAGGAGGCG AAGTAAGTAG AGGGCTGGGC TGGGCTGTGG GGGGTGTGGG |
| 5751 | GTGCGGGACT GGGCAGTCTG GGAGTCCCTC TCACCACTTT TCTTACCTTT |
| 5801 | CTAGGATGCT GCCGTCGCCG CCGCTCATAC ACCATAAGGT GTAAAAAATA |
| 5851 | CTAGATGCAC AGAATAGCAA GTCCATCAAA ACTCCTGCGT GAGAATTTTA |
| 5901 | CCAGACTTCA AGAGCATCTC GCCACATCTT GAAAAATGCC ACCGTCCGAT |
| 5951 | GAAAAACAGG AGCCTGCTAA GGAACAATGC CACCTGTCAA TAAATGTTGA |
| 6001 | AAACTCATCC CATTCCTGCC TCTTGGTCCT TGGGCTTGGG GAGGGGTGCG |
| 6051 | CGGATGTGGT TAGGGAACAT GACTGGTCAA ATGGGAAGGG CTTCAAAAGA |
| 6101 | ATTCCCAATA TTGACTACCA AGCCACCTGT ACAGATCGAA TTCAGATCTG |
| 6151 | CCTGCAGGCA TGCAAGCTTG GCACTGGCCG TCGTTTTACA ACGTCGTGAC |
| 6201 | TGGGAAAACC CTGGCGTTAC CCAACTTAAT CGCCTTGCAG CACATCCCCC |
| 6251 | TTTCGCCAGC TGGCGTAATA GCGAAGAGGC CCGCACCGAT CGCCCTTCCC |
| 6301 | AACAGTTGCG CAGCCTGAAT GGCGAATGGC GCCTGATGCG GTATTTTCTC |
| 6351 | CTTACGCATC TGTGCGGTAT TTCACACCGC ATATGGTGCA CTCTCAGTAC |
| 6401 | AATCTGCTCT GATGCCGCAT AGTTAAGCCA GCCCCGACAC CCGCCAACAC |
| 6451 | CCGCTGACGC GCCCTGACGG GCTTGTCTGC TCCCGGCATC CGCTTACAGA |
| 6501 | CAAGCTGTGA CCGTCTCCGG GAGCTGCATG TGTCAGAGGT TTTCACCGTC |
| 6551 | ATCACCGAAA CGCGCGAGAC GAAAGGGCCT CGTGATACGC CTATTTTTAT |
| 6601 | AGGTTAATGT CATGATAATA ATGGTTTCTT AGACGTCAGG TGGCACTTTT |
| 6651 | CGGGGAAATG TGCGCGGAAC CCCTATTTGT TTATTTTTCT AAATACATTC |
| 6701 | AAATATGTAT CCGCTCATGA GACAATAACC CTGATAAATG CTTCAATAAT |
| 6751 | ATTGAAAAAG GAAGAGTATG AGTATTCAAC ATTTCCGTGT CGCCCTTATT |
| 6801 | CCCTTTTTTG CGGCATTTTG CCTTCCTGTT TTTGCTCACC CAGAAACGCT |
| 6851 | GGTGAAAGTA AAAGATGCTG AAGATCAGTT GGGTGCACGA GTGGGTTACA |
| 6901 | TCGAACTGGA TCTCAACAGC GGTAAGATCC TTGAGAGTTT TCGCCCCGAA |
| 6951 | GAACGTTTTC CAATGATGAG CACTTTTAAA GTTCTGCTAT GTGGCGCGGT |
| 7001 | ATTATCCCGT ATTGACGCCG GGCAAGAGCA ACTCGGTCGC CGCATACACT |
| 7051 | ATTCTCAGAA TGACTTGGTT GAGTACTCAC CAGTCACAGA AAAGCATCTT |
| 7101 | ACGGATGGCA TGACAGTAAG AGAATTATGC AGTGCTGCCA TAACCATGAG |
| 7151 | TGATAACACT GCGGCCAACT TACTTCTGAC AACGATCGGA GGACCGAAGG |
| 7201 | AGCTAACCGC TTTTTTGCAC AACATGGGGG ATCATGTAAC TCGCCTTGAT |
| 7251 | CGTTGGGAAC CGGAGCTGAA TGAAGCCATA CCAAACGACG AGCGTGACAC |
| 7301 | CACGATGCCT GTAGCAATGG CAACAACGTT GCGCAAACTA TTAACTGGCG |
| 7351 | AACTACTTAC TCTAGCTTCC CGGCAACAAT TAATAGACTG GATGGAGGCG |
| 7401 | GATAAAGTTG CAGGACCACT TCTGCGCTCG GCCCTTCCGG CTGGCTGGTT |
| 7451 | TATTGCTGAT AAATCTGGAG CCGGTGAGCG TGGGTCTCGC GGTATCATTG |
| 7501 | CAGCACTGGG GCCAGATGGT AAGCCCTCCC GTATCGTAGT TATCTACACG |
| 7551 | ACGGGGAGTC AGGCAACTAT GGATGAACGA AATAGACAGA TCGCTGAGAT |
| 7601 | AGGTGCCTCA CTGATTAAGC ATTGGTAACT GTCAGACCAA GTTTACTCAT |
| 7651 | ATATACTTTA GATTGATTTA AAACTTCATT TTTAATTTAA AAGGATCTAG |
| 7701 | GTGAAGATCC TTTTTGATAA TCTCATGACC AAAATCCCTT AACGTGAGTT |
| 7751 | TTCGTTCCAC TGAGCGTCAG ACCCCGTAGA AAAGATCAAa GGATCTTCTT |
| 7801 | GAGATCCTTT TTTTCTGCGC GTAATCTGCT GCTTGCAAAC AAAAAAACCA |
| 7851 | CCGCTACCAG CGGTGGTTTG TTTGCCGGAT CAAGAGCTAC CAACTCTTTT |
| 7901 | TCCGAAGGTA ACTGGCTTCA GCAGAGCGCA GATACCAAAT ACTGTCCTTC |
| 7951 | TAGTGTAGCC GTAGTTAGGC CACCACTTCA AGAACTCTGT AGCACCGCCT |
| 8001 | ACATACCTCG CTCTGCTAAT CCTGTTACCA GTGGCTGCTG CCAGTGGCGA |
| 8051 | TAAGTCGTGT CTTACCGGGT TGGACTCAAG ACGATAGTTA CCGGATAAGG |
| 8101 | CGCAGCGGTC GGGCTGAACG GGGGGTTCGT GCACACAGCC CAGCTTGGAG |
| 8151 | CGAACGACCT ACACCGAACT GAGATACCTA CAGCGTGAGC TATGAGAAAG |
| 8201 | CGCCACGCTT CCCGAAGGGA GAAAGGCGGA CAGGTATCCG GTAAGCGGCA |
| 8251 | GGGTCGGAAC AGGAGAGCGC ACGAGGGAGC TTCCAGGGGG AAACGCCTGG |
| 8301 | TATCTTTATA GTCCTGTCGG GTTTCGCCAC CTCTGACTTG AGCGTCGATT |
| 8351 | TTTGTGATGC TCGTCAGGGG GGCGGAGCCT ATGGAAAAAC GCCAGCAACG |
| 8401 | CGGCCTTTTT ACGGTTCCTG GCCTTTTGCT GGCCTTTTGC TCACATGTTC |
| 8451 | TTTCCTGCGT TATCCCCTGA TTCTGTGGAT AACCGTATTA CCGCCTTTGA |
| 8501 | GTGAGCTGAT ACCGCTCGCC GCAGCCGAAC GACCGAGCGC AGCGAGTCAG |
| 8551 | TGAGCGAGGA AGCGGAAGA |

TABLE 6
pGFA EcoNI Control (Length 5346 bp)

VERSION pDRAW 1.0 beta
DNAname pGFA EcoNI Control
IScircular YES
Sequence . . .

| | |
|---|---|
| 1 | GCGCCCAATA CGCAAACCGC CTCTCCCCGC GCGTTGGCCG ATTCATTAAT |
| 51 | GCAGCTGGCA CGACAGGTTT CCCGACTGGA AAGCGGGCAG TGAGCGCAAC |
| 101 | GCAATTAATG TGAGTTAGCT CACTCATTAG GCACCCCAGG CTTTACACTT |
| 151 | TATGCTTCCG GCTCGTATGT TGTGTGGAAT TGTGAGCGGA TAACAATTTC |
| 201 | ACACAGGAAA CAGCTATGAC ATGATTACGA ATTCGAGCTC GGTACCAGAT |
| 251 | CTGAGCTCCC ACCTCCCTCT CTGTGCTGGG ACTCACAGAG GGAGACCTCA |
| 301 | GGAGGCAGTC TGTCCATCAC ATGTCCAAAT GCAGAGCATA CCCTGGGCTG |
| 351 | GGCGCAGTGG CGCACAACTG TAATTCCAGC ACTTTGGGAG GCTGATGTGG |
| 401 | AAGGATCACT TGAGCCCAGA AGTTCTAGAC CAGCCTGGGC AACATGGCAA |
| 451 | GACCCTATCT CTACAAAAAA AGTTAAAAAA TCAGCCACGT GTGGTGACAC |
| 501 | ACACCTGTAG TCCCAGCTAT TCAGGAGGCT GAGGTGAGGG GATCACTTAA |
| 551 | GGCTGGGAGG TTGAGGCTGC AGTGAGTCGT GGTTGCGCCA CTGCACTCCA |
| 601 | GCCTGGGCAA CAGTGAGACC CTGTCTCAAA AGACAAAAAA AAAAAAAAAA |
| 651 | AAAAAAAGAA CATATCCTGG TGTGGAGTAG GGGACGCTGC TCTGACAGAG |
| 701 | GCTCGGGGGC CTGAGCTGGC TCTGTGAGCT GGGGAGGAGG CAGACAGCCA |
| 751 | GGCCTTGTCT GCAAGCAGAC CTGGCAGCAT TGGGCTGGCC GCCCCCCAGG |
| 801 | GCCTCCTCTT CATGCCCAGT GAATGACTCA CCTTGGCACA GACACAATGT |
| 851 | TCGGGGTGGG CACAGTGCCT GCTTCCCGCC GCACCCCAGC CCCCCTCAAA |
| 901 | TGCCTTCCGA GAAGCCCATT GAGCAGGGGG CTTGCATTGC ACCCCAGCCT |
| 951 | GACAGCCTGG CATCTTGGGA TAAAAGCAGC ACAGCCCCCT AGGGGCTGCC |
| 1001 | CTTGCTGTGT GGCGCCACCG GCGGTGGAGA ACAAGGCTCT ATTCAGCCTG |
| 1051 | TGCCCAGGAA AGGGGATCAG GGGATGCCCA GGCATGGACA GTGGGTGGCA |
| 1101 | GGGGGGGAGA GGAGGGCTGT CTGCTTCCCA GAAGTCCAAG GACACAAATG |
| 1151 | GGTGAGGGGA CTGGGCAGGG TTCTGACCCT GTGGGACCAG AGTGGAGGGC |
| 1201 | GTAGATGGAC CTGAAGTCTC CAGGGACAAC AGGGCCCAGG TCTCAGGCTC |
| 1251 | CTAGTTGGGC CCAGTGGCTC CAGCGTTTCC AAACCCATCC ATCCCCAGAG |
| 1301 | GTTCTTCCCA TCTCTCCAGG CTGATGTGTG GGAACTCGAG GAAATAAATC |
| 1351 | TCCAGTGGGA GACGGAGGGG TGGCCAGGGA AACGGGGCGC TGCAGGAATA |
| 1401 | AAGACGAGCC AGCACAGCCA GCTCATGTGT AACGGCTTTG TGGAGCTGTC |
| 1451 | AAGGCCTGGT CTCTGGGAGA GAGGCACAGG GAGGCCAGAC AAGGAAGGGG |
| 1501 | TGACCTGGAG GGACAGATCC AGGGGCTAAA GTCCTGATAA GGCAAGAGAG |
| 1551 | TGCCGGCCCC CTCTTGCCCT ATCAGGACCT CCACTGCCAC ATAGAGGCCA |
| 1601 | TGATTGACCC TTAGACAAAG GGCTGGTGTC CAATCCCAGC CCCCAGCCCC |
| 1651 | AGAACTCCAG GGAATGAATG GGCAGAGAGC AGGAATGTGG GACATCTGTG |
| 1701 | TTCAAGGGAA GGACTCCAGG AGTCTGCTGG GAATGAGGCC TAGTAGGAAA |
| 1751 | TGAGGTGGCC CTTGAGGGTA CAGAACAGGT TCATTCTTCG CCAAATTCCC |
| 1801 | AGCACCTTGC AGGCACTTAC AGCTGAGTGA GATAATGCCT GGGTTATGAA |
| 1851 | ATCAAAAAGT TGGAAAGCAG GTCAGAGGTC ATCTGGTACA GCCCTTCCTT |
| 1901 | CCCTTTTTTT TTTTTTTTTT TTGTGAGACA AGGTCTCTCT CTGTTGCCCA |
| 1951 | GGCTGGAGTG GCGCAAACAC AGCTCACTGC AGCCTCAACC TACTGGGCTC |
| 2001 | AAGCAATCCT CCAGCCTCAG CCTCCCAAAG TGCTGGGATT ACAAGCATGA |
| 2051 | GCCACCCCAC TCAGCCCTTT CCTTCCTTTT TAATTGATGC ATAATAATTG |
| 2101 | TAAGTATTCA TCATGGTCCA ACCAACCCTT TCTTGACCCA CCTTCCTAGA |
| 2151 | GAGAGGGTCC TCTTGCTTCA GCGGTCAGGG CCCCAGACCC ATGGTCTGGC |
| 2201 | TCCAGGTACC ACCTGCCTCA TAAAAAACAG CACAAAAGGA AACTCACCCT |
| 2251 | AACTGTAAAG TAATTGTGTG TTTTGAGACT ATAAATATCC CTTGGAGAAA |
| 2301 | AGCCTTGTTT GGGCCCCCCC TCGAGGTCGA CGGTATCGAT AAGCTTGATA |
| 2351 | TCGAATTCCT GCAGCCCGGG GGATCCGCAG ATCCCGGCCA GATACCGATG |
| 2401 | CTGCCGCAGC AAAAGCAGGA GCAGATGCCG CCGTCGCAGG CGAAGATGTC |
| 2451 | GCAGACGGAG GAGGCGATGC TGCCGGCGGA GGAGGCGAAG TAAGTAGAGG |
| 2501 | GCTGGGCTGG GCTGTGGGGG GTGTGGGGTG CGGGACTGGG CAGTCTGGGA |
| 2551 | GTCCCTCTCA CCACTTTTCT TACCTTTCTA GGATGCTGCC GTCGCCGCCG |
| 2601 | CTCATACACC ATAAGGTGTA AAAAATACTA GATGCACAGA ATAGCAAGTC |
| 2651 | CATCAAAACT CCTGCGTGAG AATTTTACCA GACTTCAAGA GCATCTCGCC |
| 2701 | ACATCTTGAA AAATGCCACC GTCCGATGAA AAACAGGAGC CTGCTAAGGA |
| 2751 | ACAATGCCAC CTGTCAATAA ATGTTGAAAA CTCATCCCAT TCCTGCCTCT |
| 2801 | TGGTCCTTGG GCTTGGGGAG GGGTGCGCGG ATGTGGTTAG GGAACATGAC |
| 2851 | TGGTCAAATG GGAAGGGCTT CAAAAGAATT CCCAATATTG ACTACCAAGC |
| 2901 | CACCTGTACA GATCGAATTC AGATCTGCCT GCAGGCATGC AAGCTTGGCA |
| 2951 | CTGGCCGTCG TTTTACAACG TCGTGACTGG GAAAACCCTG GCGTTACCCA |
| 3001 | ACTTAATCGC CTTGCAGCAC ATCCCCCTTT CGCCAGCTGG CGTAATAGCG |
| 3051 | AAGAGGCCCG CACCGATCGC CCTTCCCAAC AGTTGCGCAG CCTGAATGGC |
| 3101 | GAATGGCGCC TGATGCGGTA TTTTCTCCTT ACGCATCTGT GCGGTATTTC |
| 3151 | ACACCGCATA TGGTGCACTC TCAGTACAAT CTGCTCTGAT GCCGCATAGT |
| 3201 | TAAGCCAGCC CCGACACCCG CCAACACCCG CTGACGCGCC CTGACGGGCT |
| 3251 | TGTCTGCTCC CGGCATCCGC TTACAGACAA GCTGTGACCG TCTCCGGGAG |
| 3301 | CTGCATGTGT CAGAGGTTTT CACCGTCATC ACCGAAACGC GCGAGACGAA |
| 3351 | AGGGCCTCGT GATACGCCTA TTTTTATAGG TTAATGTCAT GATAATAATG |
| 3401 | GTTTCTTAGA CGTCAGGTGG CACTTTTCGG GGAAATGTGC GCGGAACCCC |
| 3451 | TATTTGTTTA TTTTTCTAAA TACATTCAAA TATGTATCCG CTCATGAGAC |
| 3501 | AATAACCCTG ATAAATGCTT CAATAATATT GAAAAAGGAA GAGTATGAGT |
| 3551 | ATTCAACATT TCCGTGTCGC CCTTATTCCC TTTTTTGCGG CATTTTGCCT |
| 3601 | TCCTGTTTTT GCTCACCCAG AAACGCTGGT GAAAGTAAAA GATGCTGAAG |
| 3651 | ATCAGTTGGG TGCACGAGTG GGTTACATCG AACTGGATCT CAACAGCGGT |
| 3701 | AAGATCCTTG AGAGTTTTCG CCCCGAAGAA CGTTTTCCAA TGATGAGCAC |
| 3751 | TTTTAAAGTT CTGCTATGTG GCGCGGTATT ATCCCGTATT GACGCCGGGC |
| 3801 | AAGAGCAACT CGGTCGCCGC ATACACTATT CTCAGAATGA CTTGGTTGAG |
| 3851 | TACTCACCAG TCACAGAAAA GCATCTTACG GATGGCATGA CAGTAAGAGA |
| 3901 | ATTATGCAGT GCTGCCATAA CCATGAGTGA TAACACTGCG GCCAACTTAC |
| 3951 | TTCTGACAAC GATCGGAGGA CCGAAGGAGC TAACCGCTTT TTTGCACAAC |
| 4001 | ATGGGGGATC ATGTAACTCG CCTTGATCGT TGGGAACCGG AGCTGAATGA |
| 4051 | AGCCATACCA AACGACGAGC GTGACACCAC GATGCCTGTA GCAATGGCAA |
| 4101 | CAACGTTGCG CAAACTATTA ACTGGCGAAC TACTTACTCT AGCTTCCCGG |
| 4151 | CAACAATTAA TAGACTGGAT GGAGGCGGAT AAAGTTGCAG GACCACTTCT |
| 4201 | GCGCTCGGCC CTTCCGGCTG GCTGGTTTAT TGCTGATAAA TCTGGAGCCG |
| 4251 | GTGAGCGTGG GTCTCGCGGT ATCATTGCAG CACTGGGGCC AGATGGTAAG |
| 4301 | CCCTCCCGTA TCGTAGTTAT CTACACGACG GGGAGTCAGG CAACTATGGA |
| 4351 | TGAACGAAAT AGACAGATCG CTGAGATAGG TGCCTCACTG ATTAAGCATT |
| 4401 | GGTAACTGTC AGACCAAGTT TACTCATATA TACTTTAGAT TGATTTAAAA |
| 4451 | CTTCATTTTT AATTTAAAAG GATCTAGGTG AAGATCCTTT TTGATAATCT |
| 4501 | CATGACCAAA ATCCCTTAAC GTGAGTTTTC GTTCCACTGA GCGTCAGACC |
| 4551 | CCGTAGAAAA GATCAAAGGA TCTTCTTGAG ATCCTTTTTT TCTGCGCGTA |
| 4601 | ATCTGCTGCT TGCAAACAAA AAAACCACCG CTACCAGCGG TGGTTTGTTT |
| 4651 | GCCGGATCAA GAGCTACCAA CTCTTTTTCC GAAGGTAACT GGCTTCAGCA |
| 4701 | GAGCGCAGAT ACCAAATACT GTCCTTCTAG TGTAGCCGTA GTTAGGCCAC |
| 4751 | CACTTCAAGA ACTCTGTAGC ACCGCCTACA TACCTCGCTC TGCTAATCCT |
| 4801 | GTTACCAGTG GCTGCTGCCA GTGGCGATAA GTCGTGTCTT ACCGGGTTGG |
| 4851 | ACTCAAGACG ATAGTTACCG GATAAGGCGC AGCGGTCGGG CTGAACGGGG |
| 4901 | GGTTCGTGCA CACAGCCCAG CTTGGAGCGA ACGACCTACA CCGAACTGAG |
| 4951 | ATACCTACAG CGTGAGCTAT GAGAAAGCGC CACGCTTCCC GAAGGGAGAA |
| 5001 | AGGCGGACAG GTATCCGGTA AGCGGCAGGG TCGGAACAGG AGAGCGCACG |
| 5051 | AGGGAGCTTC CAGGGGGAAA CGCCTGGTAT CTTTATAGTC CTGTCGGGTT |
| 5101 | TCGCCACCTC TGACTTGAGC GTCGATTTTT GTGATGCTCG TCAGGGGGGC |
| 5151 | GGAGCCTATG GAAAAACGCC AGCAACGCGG CCTTTTTACG GTTCCTGGCC |
| 5201 | TTTTGCTGGC CTTTTGCTCA CATGTTCTTT CCTGCGTTAT CCCCTGATTC |
| 5251 | TGTGGATAAC CGTATTACCG CCTTTGAGTG AGCTGATACC GCTCGCCGCA |
| 5301 | GCCGAACGAC CGAGCGCAGC GAGTCAGTGA GCGAGGAAGC GGAAGA |

TABLE 7
pGFA SmaI Control (Length 5185 pb)

VERSION pDRAW 1.0 beta
DNAname pGFA SmaI Control
IScircular YES
Sequence . . .

| | |
|---|---|
| 1 | GCGCCCAATA CGCAAACCGC CTCTCCCCGC GCGTTGGCCG ATTCATTAAT |
| 51 | GCAGCTGGCA CGACAGGTTT CCCGACTGGA AAGCGGGCAG TGAGCGCAAC |
| 101 | GCAATTAATG TGAGTTAGCT CACTCATTAG GCACCCCAGG CTTTACACTT |
| 151 | TATGCTTCCG GCTCGTATGT TGTGTGGAAT TGTGAGCGGA TAACAATTTC |
| 201 | ACACAGGAAA CAGCTATGAC ATGATTACGA ATTCGAGCTC GGTACCAGAT |
| 251 | CTGAGCTCCC ACCTCCCTCT CTGTGCTGGG ACTCACAGAG GGAGACCTCA |
| 301 | GGAGGCAGTC TGTCCATCAC ATGTCCAAAT GCAGAGCATA CCCTGGGCTG |
| 351 | GGCGCAGTGG CGCACAACTG TAATTCCAGC ACTTTGGGAG GCTGATGTGG |
| 401 | AAGGATCACT TGAGCCCAGA AGTTCTAGAC CAGCCTGGGC AACATGGCAA |
| 451 | GACCCTATCT CTACAAAAAA AGTTAAAAAA TCAGCCACGT GTGGTGACAC |
| 501 | ACACCTGTAG TCCCAGCTAT TCAGGAGGCT GAGGTGAGGG GATCACTTAA |
| 551 | GGCTGGGAGG TTGAGGCTGC AGTGAGTCGT GGTTGCGCCA CTGCACTCCA |
| 601 | GCCTGGGCAA CAGTGAGACC CTGTCTCAAA AGACAAAAAA AAAAAAAAAA |
| 651 | AAAAAAAGAA CATATCCTGG TGTGGAGTAG GGGACGCTGC TCTGACAGAG |
| 701 | GCTCGGGGGC CTGAGCTGGC TCTGTGAGCT GGGGAGGAGG CAGACAGCCA |
| 751 | GGCCTTGTCT GCAAGCAGAC CTGGCAGCAT TGGGCTGGCC GCCCCCCAGG |
| 801 | GCCTCCTCTT CATGCCCAGT GAATGACTCA CCTTGGCACA GACACAATGT |
| 851 | TCGGGGTGGG CACAGTGCCT GCTTCCCGCC GCACCCCAGC CCCCCTCAAA |
| 901 | TGCCTTCCGA GAAGCCCATT GAGCAGGGGG CTTGCATTGC ACCCCAGCCT |
| 951 | GACAGCCTGG CATCTTGGGA TAAAAGCAGC ACAGCCCCCT AGGGGCTGCC |
| 1001 | CTTGCTGTGT GGCGCCACCG GCGGTGGAGA ACAAGGCTCT ATTCAGCCTG |
| 1051 | TGCCCAGGAA AGGGGATCAG GGGATGCCCA GGCATGGACA GTGGGTGGCA |
| 1101 | GGGGGGGAGA GGAGGGCTGT CTGCTTCCCA GAAGTCCAAG GACACAAATG |
| 1151 | GGTGAGGGGA CTGGGCAGGG TTCTGACCCT GTGGGACCAG AGTGGAGGGC |
| 1201 | GTAGATGGAC CTGAAGTCTC CAGGGACAAC AGGGCCCAGG TCTCAGGCTC |
| 1251 | CTAGTTGGGC CCAGTGGCTC CAGCGTTTCC AAACCCATCC ATCCCCAGAG |
| 1301 | GTTCTTCCCA TCTCTCCAGG CTGATGTGTG GGAACTCGAG GAAATAAATC |
| 1351 | TCCAGTGGGA GACGGAGGGG TGGCCAGGGA AACGGGGCGC TGCAGGAATA |
| 1401 | AAGACGAGCC AGCACAGCCA GCTCATGTGT AACGGCTTTG TGGAGCTGTC |
| 1451 | AAGGCCTGGT CTCTGGGAGA GAGGCACAGG GAGGCCAGAC AAGGAAGGGG |
| 1501 | TGACCTGGAG GGACAGATCC AGGGGCTAAA GTCCTGATAA GGCAAGAGAG |
| 1551 | TGCCGGCCCC CTCTTGCCCT ATCAGGACCT CCACTGCCAC ATAGAGGCCA |
| 1601 | TGATTGACCC TTAGACAAAG GGCTGGTGTC CAATCCCAGC CCCCAGCCCC |
| 1651 | AGAACTCCAG GGAATGAATG GGCAGAGAGC AGGAATGTGG GACATCTGTG |
| 1701 | TTCAAGGGAA GGACTCCAGG AGTCTGCTGG GAATGAGGCC TAGTAGGAAA |
| 1751 | TGAGGTGGCC CTTGAGGGTA CAGAACAGGT TCATTCTTCG CCAAATTCCC |
| 1801 | AGCACCTTGC AGGCACTTAC AGCTGAGTGA GATAATGCCT GGGTTATGAA |
| 1851 | ATCAAAAAGT TGGAAAGCAG GTCAGAGGTC ATCTGGTACA GCCCTTCCTT |
| 1901 | CCCTTTTTTT TTTTTTTTTT TTGTGAGACA AGGTCTCTCT CTGTTGCCCA |
| 1951 | GGCTGGAGTG GCGCAAACAC AGCTCACTGC AGCCTCAACC TACTGGGCTC |
| 2001 | AAGCAATCCT CCAGCCTCAG CCTCCCAAAG TGCTGGGATT ACAAGCATGA |
| 2051 | GCCACCCCAC TCAGCCCTTT CCTTCCTTTT TAATTGATGC ATAATAATTG |
| 2101 | TAAGTATTCA TCATGGTCCA ACCAACCCTT TCTTGACCCA CCTTCCTAGA |
| 2151 | GAGAGGGTCC TCTTGCTTCA GCGGTCAGGG CCCCAGACCC ATGGTCTGGC |
| 2201 | TCCAGGTACC ACCTGCCTCA TGCAGGAGTT GGCGTGCCCA GGAAGCTCTG |
| 2251 | CCTCTGGGCA CAGTGACCTC AGTGGGGTGA GGGGAGCTCT CCCCATAGCT |
| 2301 | GGGCTGCGGC CCAACCCCAC CCCCTCAGGC TATGCCAGGG GGTGTTGCCA |
| 2351 | GGGGCACCCT AAAAAACAGC ACAAAAGGAA ACTCACCCTA ACTGTAAAGT |
| 2401 | AATTGTGTGT TTTGAGACTA TAAATATCCC TTGGAGAAAA GCCTTGTTTG |
| 2451 | GGCCCCCCCT CGAGGTCGAC GGTATCGATA AGCTTGATAT CGAATTCCTG |
| 2501 | CAGCCCGGGG GATCCGCAGA TCCCGGCCAG ATACCGATGC TGCCGCAGCA |
| 2551 | AAAGCAGGAG CAGATGCCGC CGTCGCAGGC GAAGATGTCG CAGACGGAGG |
| 2601 | AGGCGATGCT GCCGGCGGAG GAGGCGAAGT AAGTAGAGGG CTGGGCTGGG |
| 2651 | CTGTGGGGGG TGTGGGGTGC GGGACTGGGC AGTCTGGGAG TCCCTCTCAC |
| 2701 | CACTTTTCTT ACCTTTCTAG GATGCTGCCG TCGCCGCCGC TCATACACCA |
| 2751 | TAAGGTGTAA AAAATACTAG ATGCACAGAA TAGCAAGTCC ATCAAAACTC |
| 2801 | CTGCGTGAGA ATTTTACCAG ACTTCAAGAG CATCTCGCCA CATCTTGAAA |
| 2851 | AATGCCACCG TCCGATGAAA AACAGGAGCC TGCTAAGGAA CAATGCCACC |
| 2901 | TGTCAATAAA TGTTGAAAAC TCATCCCATT CCTGCCTCTT GGTCCTTGGG |
| 2951 | CTTGGGGAGG GGTGCGCGGA TGTGGTTAGG GAACATGACT GGTCAAATGG |
| 3001 | GAAGGGCTTC AAAAGAATTC CCAATATTGA CTACCAAGCC ACCTGTACAG |
| 3051 | ATCGAATTCA GATCTGCCTG CAGGCATGCA AGCTTGGCAC TGGCCGTCGT |
| 3101 | TTTACAACGT CGTGACTGGG AAAACCCTGG CGTTACCCAA CTTAATCGCC |
| 3151 | TTGCAGCACA TCCCCCTTTC GCCAGCTGGC GTAATAGCGA AGAGGCCCGC |
| 3201 | ACCGATCGCC CTTCCCAACA GTTGCGCAGC CTGAATGGCG AATGGCGCCT |
| 3251 | GATGCGGTAT TTTCTCCTTA CGCATCTGTG CGGTATTTCA CACCGCATAT |
| 3301 | GGTGCACTCT CAGTACAATC TGCTCTGATG CCGCATAGTT AAGCCAGCCC |
| 3351 | CGACACCCGC CAACACCCGC TGACGCGCCC TGACGGGCTT GTCTGCTCCC |
| 3401 | GGCATCCGCT TACAGACAAG CTGTGACCGT CTCCGGGAGC TGCATGTGTC |
| 3451 | AGAGGTTTTC ACCGTCATCA CCGAAACGCG CGAGACGAAA GGGCCTCGTG |
| 3501 | ATACGCCTAT TTTTATAGGT TAATGTCATG ATAATAATGG TTTCTTAGAC |
| 3551 | GTCAGGTGGC ACTTTTCGGG GAAATGTGCG CGGAACCCCT ATTTGTTTAT |
| 3601 | TTTTCTAAAT ACATTCAAAT ATGTATCCGC TCATGAGACA ATAACCCTGA |
| 3651 | TAAATGCTTC AATAATATTG AAAAAGGAAG AGTATGAGTA TTCAACATTT |
| 3701 | CCGTGTCGCC CTTATTCCCT TTTTTGCGGC ATTTTGCCTT CCTGTTTTTG |
| 3751 | CTCACCCAGA AACGCTGGTG AAAGTAAAAG ATGCTGAAGA TCAGTTGGGT |
| 3801 | GCACGAGTGG GTTACATCGA ACTGGATCTC AACAGCGGTA AGATCCTTGA |
| 3851 | GAGTTTTCGC CCCGAAGAAC GTTTTCCAAT GATGAGCACT TTTAAAGTTC |
| 3901 | TGCTATGTGG CGCGGTATTA TCCCGTATTG ACGCCGGGCA AGAGCAACTC |
| 3951 | GGTCGCCGCA TACACTATTC TCAGAATGAC TTGGTTGAGT ACTCACCAGT |
| 4001 | CACAGAAAAG CATCTTACGG ATGGCATGAC AGTAAGAGAA TTATGCAGTG |
| 4051 | CTGCCATAAC CATGAGTGAT AACACTGCGG CCAACTTACT TCTGACAACG |
| 4101 | ATCGGAGGAC CGAAGGAGCT AACCGCTTTT TTGCACAACA TGGGGGATCA |
| 4151 | TGTAACTCGC CTTGATCGTT GGGAACCGGA GCTGAATGAA GCCATACCAA |
| 4201 | ACGACGAGCG TGACACCACG ATGCCTGTAG CAATGGCAAC AACGTTGCGC |
| 4251 | AAACTATTAA CTGGCGAACT ACTTACTCTA GCTTCCCGGC AACAATTAAT |
| 4301 | AGACTGGATG GAGGCGGATA AAGTTGCAGG ACCACTTCTG CGCTCGGCCC |
| 4351 | TTCCGGCTGG CTGGTTTATT GCTGATAAAT CTGGAGCCGG TGAGCGTGGG |
| 4401 | TCTCGCGGTA TCATTGCAGC ACTGGGGCCA GATGGTAAGC CCTCCCGTAT |
| 4451 | CGTAGTTATC TACACGACGG GGAGTCAGGC AACTATGGAT GAACGAAATA |
| 4501 | GACAGATCGC TGAGATAGGT GCCTCACTGA TTAAGCATTG GTAACTGTCA |
| 4551 | GACCAAGTTT ACTCATATAT ACTTTAGATT GATTTAAAAC TTCATTTTTA |
| 4601 | ATTTAAAAGG ATCTAGGTGA AGATCCTTTT TGATAATCTC ATGACCAAAA |
| 4651 | TCCCTTAACG TGAGTTTTCG TTCCACTGAG CGTCAGACCC CGTAGAAAAG |
| 4701 | ATCAAAGGAT CTTCTTGAGA TCCTTTTTTT CTGCGCGTAA TCTGCTGCTT |
| 4751 | GCAAACAAAA AAACCACCGC TACCAGCGGT GGTTTGTTTG CCGGATCAAG |
| 4801 | AGCTACCAAC TCTTTTTCCG AAGGTAACTG GCTTCAGCAG AGCGCAGATA |
| 4851 | CCAAATACTG TCCTTCTAGT GTAGCCGTAG TTAGGCCACC ACTTCAAGAA |
| 4901 | CTCTGTAGCA CCGCCTACAT ACCTCGCTCT GCTAATCCTG TTACCAGTGG |
| 4951 | CTGCTGCCAG TGGCGATAAG TCGTGTCTTA CCGGGTTGGA CTCAAGACGA |
| 5001 | TAGTTACCGG ATAAGGCGCA GCGGTCGGGC TGAACGGGGG GTTCGTGCAC |
| 5051 | ACAGCCCAGC TTGGAGCGAA CGACCTACAC CGAACTGAGA TACCTACAGC |
| 5101 | GTGAGCTATG AGAAAGCGCC ACGCTTCCCG AAGGGAGAAA GGCGGACAGG |
| 5151 | TATCCGGTAA GCGGCAGGGT CGGAACAGGA GAGCGCACGA GGGAGCTTCC |
| 5201 | AGGGGGAAAC GCCTGGTATC TTTATAGTCC TGTCGGGTTT CGCCACCTCT |
| 5251 | GACTTGAGCG TCGATTTTTG TGATGCTCGT CAGGGGGGCG GAGCCTATGG |
| 5301 | AAAAACGCCA GCAACGCGGC CTTTTTACGG TTCCTGGCCT TTTGCTGGCC |
| 5351 | TTTTGCTCAC ATGTTCTTTC CTGCGTTATC CCCTGATTCT GTGGATAACC |
| 5401 | GTATTACCGC CTTTGAGTGA GCTGATACCG CTCGCCGCAG CCGAACGACC |
| 5451 | GAGCGCAGCG AGTCAGTGAG CGAGGAAGCG GAAGA |

1. A nucleic acid construct comprising a Pol III/Pol II fusion promoter comprising
an RNA Polymerase III-binding basal promoter region; and
cis-acting regulatory regions from a Pol II promoter, operably linked with said basal promoter region;
wherein said cis-acting regulatory regions provide specific regulation of expression from said construct.

2. The construct of claim 1, wherein said cis-acting regulatory regions provide cell-specific regulation, tissue-specific regulation, cell-cycle specific regulation, tumor-specific regulation in vivo, radiation-induced expression in vivo, or estrogen-induced expression in vivo.

3-7. (canceled)

8. The construct of claim 1, wherein said construct further comprises a sequence encoding a shRNA, a siRNA, or an RNAi agent targeting an mRNA of a disease-associated gene, wherein said sequence is operably linked with said fusion promoter.

9. (canceled)

10. The construct of claim 1, wherein said basal promoter region is selected from the group consisting of a Pol III basal promoter, a U6 basal promoter, a H1 basal promoter, a tRNA basal promoter, and a mutated Pol II basal promoter, wherein said mutated Pol II basal promoter preferentially binds Pol III instead of Pol II.

11-14. (canceled)

15. The construct of claim 1, wherein said Pol II cis-acting regulatory regions comprise CMV early intermediate regulatory regions or a complete Pol II regulatory region less the basal promoter.

16-17. (canceled)

18. A vector comprising a Pol III/Pol II fusion promoter comprising
a RNA Polymerase III-binding basal promoter region; and
cis-acting regulatory regions from a Pol II promoter operably linked with said basal promoter region;
wherein said cis-acting regulatory regions allow specific regulation of expression from said fusion promoter.

19. The vector of claim 18, further comprising a sequence encoding an RNAi agent, a siRNA, or a shRNA, wherein said sequence is operably linked with said fusion promoter.

20-21. (canceled)

22. The vector of claim 18, wherein said vector is selected from the group consisting of a plasmid, a viral-based vector, a replication defective vector, and a replication competent vector.

23-25. (canceled)

26. A cell comprising a Pol III/Pol II fusion promoter operably linked with a coding sequence, wherein said fusion promoter comprises
a RNA Polymerase III-binding basal promoter region; and
cis-acting regulatory regions from a Pol II promoter operably linked with said basal promoter region;
wherein said cis-acting regulatory regions allow specific regulation of expression from said fusion promoter.

27. The cell of claim 26, wherein said coding sequence encodes an RNAi agent, a siRNA, or a shRNA.

28-29. (canceled)

30. The cell of claim 27, wherein said fusion promoter and said RNAi agent are in a vector.

31. The cell of claim 30, wherein said vector is a plasmid or a viral vector.

32. (canceled)

33. The cell of claim 26, wherein said cell is in cell culture.

34. The cell of claim 33, wherein said cell is selected from the group consisting of an animal cell, a human cell, an insect cell, and a plant cell.

35-37. (canceled)

38. The cell of claim 26, wherein said cell is in an animal, a plant, or a fungus.

39. The cell of claim 38, wherein said animal is selected from the group consisting of a human, a bovine, a porcine, an ovine, a feline, a canine, and a bird.

40-47. (canceled)

48. A non-human transgenic organism, comprising
a plurality of cells comprising a genetic construct comprising a Pol III/Pol II fusion promoter operably linked with a coding sequence, wherein said Pol III/Pol II fusion promoter comprises
a RNA Polymerase III-binding basal promoter region; and
cis-acting regulatory regions from a Pol II promoter operably linked with said basal promoter region;
wherein said cis-acting regulatory regions allow specific regulation of expression from said fusion promoter.

49. The organism of claim 48, wherein said coding sequence encodes an RNAi agent.

50. The organism of claim 48, wherein said organism is an animal or a plant.

51-55. (canceled)

56. A pharmaceutical composition comprising a nucleic acid construct of claim 1, wherein said nucleic acid construct further comprises a shRNA or siRNA sequence operatively linked with said fusion promoter; and
a pharmaceutically acceptable carrier or excipient.

57. The pharmaceutical composition of claim 56, wherein said composition is formulated as an injectable composition, a composition for topical administration, or a liposomal composition.

58-59. (canceled)

60. The pharmaceutical composition of claim 56, wherein said composition comprises a viral vector.

61-64. (canceled)

65. A method for expressing an RNAi agent in a cell, comprising
maintaining a cell under expression conditions, wherein said cell comprises a genetic construct comprising a Pol III/Pol II fusion promoter operably linked with a RNAi agent encoding sequence, wherein said Pol III/Pol II fusion promoter comprises
a RNA Polymerase III-binding basal promoter region; and
cis-acting regulatory regions from a Pol II promoter, operably linked with said basal promoter region;
wherein said cis-acting regulatory regions allow specific regulation of expression from said fusion promoter.

66. The method of claim 65, wherein said RNAi agent is an shRNA or an siRNA.

67. (canceled)

68. A method for inhibiting expression of a target gene in a cell, comprising
transfecting said cell with a vector comprising a genetic construct, wherein said construct comprises a Pol III/Pol II fusion promoter operably linked with a nucleic acid sequence encoding an RNAi agent targeted to said target gene, wherein said fusion promoter comprises
a RNA Polymerase III-binding basal promoter region; and cis-acting regulatory regions from a Pol II promoter, operably linked with said basal promoter region, wherein said cis-acting regulatory regions allow specific regulation of expression from said fusion promoter; and
maintaining said cell under expression conditions.

69. The method of claim 68, wherein said cell is in an organism.

70. The method of claim 68, wherein said construct comprises a regulatory element selected from the group consisting of a tissue-specific regulatory element and a tumor-specific regulatory element wherein said target gene is preferentially inhibited in said cell corresponding to said regulatory element.

71. (canceled)

72. The method of claim 68, wherein said inhibition is induced in response to radiation, the presence of an effective amount of a non-peptide and non-nucleotidic chemical species, or an estrogen.

73-79. (canceled)

80. A method for validating a target as a therapeutic target, comprising
inhibiting expression of a putative therapeutic target gene in said cell, wherein said inhibiting is due to expression of an RNAi agent from a genetic construct comprising a Pol III/Pol II fusion promoter operably linked with a nucleic acid sequence encoding said RNAi agent, wherein said fusion promoter comprises a RNA Polymerase III-binding basal promoter region; and cis-acting regulatory regions from a Pol II promoter operably linked with said basal promoter region, wherein said cis-acting regulatory regions allow specific regulation of expression from said fusion promoter; and
determining whether a biological change in said cell following said inhibiting corresponds with a therapeutic effect, wherein correspondence of said biological change with said therapeutic effect is indicative that said gene is a therapeutic target gene.

81-83. (canceled)

84. A method for treating a disease or condition wherein inhibition of a target gene provides a beneficial effect, comprising
administering a pharmacologically effective amount of a vector comprising a genetic construct comprising a Pol III/Pol II fusion promoter providing specific regulation of expression, operably linked with a sequence encoding an RNAi agent, to a subject suffering from or at risk of said disease or condition,
wherein said fusion promoter comprises a RNA Polymerase III-binding basal promoter region; and cis-acting regulatory regions from a Pol II promoter operably linked with said basal promoter region, wherein said cis-acting regulatory regions provide specific regulation of expression from said fusion promoter.

85. The method of claim 84, wherein said vector is a plasmid or a viral vector.

86. (canceled)

87. The method of claim 84, wherein said subject is selected from the group consisting of a human, a non-human animal, and a plant.

88-89. (canceled)

90. The method of claim 84, wherein said RNAi agent is an shRNA or an siRNA.

91-101. (canceled)


SEQUENCE LISTING (7 sequences)

SEQ ID NO: 1 - DNA, 3292 bp, Artificial Sequence
  pSilencer 1.0
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacccgctctagaactagtggatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggannttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 2 - DNA, 2129 bp, Artificial Sequence
  Synthetic construct
agatctgagctcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctctaaaaaacagcacaaaaggaaactcaccctaactgtaaagtaattgtgtgttttgagactataaatatcccttggagaaaagccttgtttgggccccccctcgaggtcgacggtatcgataagcttgatatcgaattcctgcagcccgggggatcc

SEQ ID NO: 3 - DNA, 2269 bp, Artificial Sequence
  Synthetic construct
agatctgagctcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcaccctaaaaaacagcacaaaaggaaactcaccctaactgtaaagtaattgtgtgttttgagactataaatatcccttggagaaaagccttgtttgggccccccctcgaggtcgacggtatcgataagcttgatatcgaattcctgcagcccgggggatcc

SEQ ID NO: 4 - DNA, 6710 bp, Artificial Sequence
  Expression vector  peGFP-HcRed1
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctaggggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagtcgccaccatggtgagcggcctgctgaaggagagtatgcgcatcaagatgtacatggagggcaccgtgaacggccactacttcaagtgcgagggcgagggcgacggcaaccccttcgccggcacccagagcatgagaatccacgtgaccgagggcgcccccctgcccttcgccttcgacatcctggccccctgctgcgagtacggcagcaggaccttcgtgcaccacaccgccgagatccccgacttcttcaagcagagcttccccgagggcttcacctgggagagaaccaccacctacgaggacggcggcatcctgaccgcccaccaggacaccagcctggagggcaactgcctgatctacaaggtgaaggtgcacggcaccaacttccccgccgacggccccgtgatgaagaacaagagcggcggctgggagcccagcaccgaggtggtgtaccccgagaacggcgtgctgtgcggccggaacgtgatggccctgaaggtgggcgaccggcacctgatctgccaccactacaccagctaccggagcaagaaggccgtgcgcgccctgaccatgcccggcttccacttcaccgacatccggctccagatgctgcggaagaagaaggacgagtacttcgagctgtacgaggccagcgtggcccggtacagcgacctgcccgagaaggccaactgaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 5 - DNA, 8569 bp, Artificial Sequence
  Synthetic construct
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtaccagatctgagctcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcggggatcctctagagtcgacggatccggggaattccccagtctcaggatccaccatgggggatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatccgcagatcccggccagataccgatgctgccgcagcaaaagcaggagcagatgccgccgtcgcaggcgaagatgtcgcagacggaggaggcgatgctgccggcggaggaggcgaagtaagtagagggctgggctgggctgtggggggtgtggggtgcgggactgggcagtctgggagtccctctcaccacttttcttacctttctaggatgctgccgtcgccgccgctcatacaccataaggtgtaaaaaatactagatgcacagaatagcaagtccatcaaaactcctgcgtgagaattttaccagacttcaagagcatctcgccacatcttgaaaaatgccaccgtccgatgaaaaacaggagcctgctaaggaacaatgccacctgtcaataaatgttgaaaactcatcccattcctgcctcttggtccttgggcttggggaggggtgcgcggatgtggttagggaacatgactggtcaaatgggaagggcttcaaaagaattcccaatattgactaccaagccacctgtacagatcgaattcagatctgcctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 6 - DNA, 5346 bp, Artificial Sequence
  Synthetic construct
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtaccagatctgagctcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcataaaaaacagcacaaaaggaaactcaccctaactgtaaagtaattgtgtgttttgagactataaatatcccttggagaaaagccttgtttgggccccccctcgaggtcgacggtatcgataagcttgatatcgaattcctgcagcccgggggatccgcagatcccggccagataccgatgctgccgcagcaaaagcaggagcagatgccgccgtcgcaggcgaagatgtcgcagacggaggaggcgatgctgccggcggaggaggcgaagtaagtagagggctgggctgggctgtggggggtgtggggtgcgggactgggcagtctgggagtccctctcaccacttttcttacctttctaggatgctgccgtcgccgccgctcatacaccataaggtgtaaaaaatactagatgcacagaatagcaagtccatcaaaactcctgcgtgagaattttaccagacttcaagagcatctcgccacatcttgaaaaatgccaccgtccgatgaaaaacaggagcctgctaaggaacaatgccacctgtcaataaatgttgaaaactcatcccattcctgcctcttggtccttgggcttggggaggggtgcgcggatgtggttagggaacatgactggtcaaatgggaagggcttcaaaagaattcccaatattgactaccaagccacctgtacagatcgaattcagatctgcctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 7 - DNA, 5485 bp, Artificial Sequence
  Synthetic construct
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtaccagatctgagctcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcaccctaaaaaacagcacaaaaggaaactcaccctaactgtaaagtaattgtgtgttttgagactataaatatcccttggagaaaagccttgtttgggccccccctcgaggtcgacggtatcgataagcttgatatcgaattcctgcagcccgggggatccgcagatcccggccagataccgatgctgccgcagcaaaagcaggagcagatgccgccgtcgcaggcgaagatgtcgcagacggaggaggcgatgctgccggcggaggaggcgaagtaagtagagggctgggctgggctgtggggggtgtggggtgcgggactgggcagtctgggagtccctctcaccacttttcttacctttctaggatgctgccgtcgccgccgctcatacaccataaggtgtaaaaaatactagatgcacagaatagcaagtccatcaaaactcctgcgtgagaattttaccagacttcaagagcatctcgccacatcttgaaaaatgccaccgtccgatgaaaaacaggagcctgctaaggaacaatgccacctgtcaataaatgttgaaaactcatcccattcctgcctcttggtccttgggcttggggaggggtgcgcggatgtggttagggaacatgactggtcaaatgggaagggcttcaaaagaattcccaatattgactaccaagccacctgtacagatcgaattcagatctgcctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga